(19) United States
(12) Patent Application Publication
(10) Pub. No.: US 2014/0248273 A1
(43) Pub. Date: Sep. 4, 2014

(54) VACCINE BASED ON STAPHYLOCOCCAL SUPERANTIGEN-LIKE 3 PROTEIN (SSL3)

(71) Applicants: Jos van Strijp, Utrecht (NL); Carla de Haas, Utrecht (NL); Paul Vermeij, St. Anthonis (NL); Bart Bardoel, Berlin (DE)

(72) Inventors: Jos van Strijp, Utrecht (NL); Carla de Haas, Utrecht (NL); Paul Vermeij, St. Anthonis (NL); Bart Bardoel, Berlin (DE)

(21) Appl. No.: 14/343,079

(22) PCT Filed: Sep. 7, 2012
(86) PCT No.: PCT/EP2012/067500

§ 371 Date: Apr. 21, 2014

(30) Foreign Application Priority Data
Sep. 8, 2001 (EP) 11180633.7

(51) Int. Cl. A61K 39/085; A61K 39/40

(52) U.S. Cl. CPC A61K 39/085; A61K 39/40
USPC 424/139.1; 424/190.1; 424/243.1

(57) ABSTRACT

The present invention relates to the field of vaccinology, especially of vaccines against Staphylococcus aureus, for both human and veterinary application. In particular the invention relates to a Staphylococcal superantigen-like 3 (SSL3) protein or its homolog, an immunogenic fragment of either protein, for use in a vaccine against S. aureus. In addition the invention relates to vaccines, methods, and medical uses of these proteins.

The present invention relates to the field of vaccinology, especially of vaccines against Staphylococcus aureus, for both human and veterinary application.

Staphylococci are nonmotile, nonspore forming, Gram positive, facultative anaerobic cocci, belonging to the Firmicutes. Colonies on blood agar are round convex, with golden colour. Staphylococcus aureus (S. aureus) is a normal commensal of the skin and mucous membranes in humans and animals. Within a few days after birth, the skin, perineal area and sometimes the gastrointestinal tract are colonized from their environment. At older age subjects may become carriers, whereby S. aureus is most commonly found in the anterior nares. These bacteria resident in or on a carrier are considered the principle cause of opportunistic infections of wounds resulting from skin abrasions or from surgery. Because S. aureus is highly versatile, it can infect almost every tissue in a subject's body.

In humans many different diseases caused by S. aureus are known, ranging from skin abscesses, to infection of joints, internal organs like endocarditis, and vascular infection. Ultimately, these may lead to generalised infection and sepsis, even resulting in death of the patient. (Plata et al., 2009, Acta Biochem. Polon., vol. 56, p. 597).

Infections with S. aureus can be hospital acquired (nosocomial) or community acquired, and derive from contact with infected surfaces or from human or animal carriers. Therefore zoonotic transfer is a major concern. In some countries hospitals take special precautions when admitting patients that had recently been in contact with lifestock.

Occurrence of S. aureus in animals is therefore increasingly being monitored. S. aureus infection in species of veterinary relevance may vary from non-symptomatic, to opportunistic, to causing serious disease with profound effects on welfare and economics. In all cases however, chance of zoonosis is now a common concern.

Examples are: S. aureus in swine, which contributes to respiratory disease problems for the pig (Atanasova et al., 2011, Vet. J., vol. 188, p. 210), but is a significant danger for transfer of the multiply antibiotic resistant strain ST398 (Pletinckx et al., 2011, Infect. Genet. Evol., in press 10.1016; Anonymous, Science 2007, vol. 329, p. 1010).

In chickens, S. aureus infection causes skeletal problems such as arthritis, tendonitis, and bone deformation, called: bacterial chondronecrosis, or femoral head necrosis, which is the leading cause of lameness in poultry. When sampling the most prominent sites for S. aureus residence in chickens, the nares and cloaca, only occasionally MRSA are found. (Joiner et al., 2005, Vet. Pathol., vol. 42, p. 275; Nemati et al., 2009, Av. Pathol., vol. 38, p. 513). However, losses occur in these cases from the locomotory problems and inability to get to the feed, especially in heavier poultry breeds.

In companion animals such as horses, cats and dogs, that are carriers, occasional opportunistic infections occur of skin, ears, or upon surgical procedures. Occasionally MRSA are encountered, now screening activity is enhanced (Faires et al., 2010, Emerg. Infect. Dis., vol. 16, p. 69; Weese et al., 2007, Canad. Vet. J., vol. 48, p. 921).

However, the main economically relevant veterinary manifestation of S. aureus infection is the infection of the mammary gland of dairy cows. This bovine mastitis leads to welfare problems from infection, but also to severe economic losses from the reduction in the quality of the milk; on the one hand because the decrease in fat and protein level reduce the milk's value, and on the other because the udder infection causes enhanced somatic cell counts in the milk, which can lead to rejection of the milk at the factory. Also the reduced quantity of milk produced is a loss. The most relevant pathogens in mastitis are S. aureus, Escherichia coli, and Streptococcus uberis. While E. coli generates a rapid inflammation of short duration; the infection of S. aureus often is subclinical. The main problem with mastitis from S. aureus is the development of chronic infection, when S. aureus may go into biofilms, or go intracellular as small-colony variant. In this late chronic stage of mastitis cows may never fully recover, and then need to be culled. (Petzl et al., 2008, Vet. Res., vol. 39, p. 18). The molecular mechanism why the infection with S. aureus could remain subclinical initially, was not understood, but a role of the innate immune system was suspected.

Current therapy for mastitis comprises the intra-mammary application of a combination of hormones and antibiotics, as vaccinations are not universally effective. (Middleton et al., 2009, Vet. Microbiol., vol. 134, p. 192; Hoogeveen et al., 2011, New Zeal. Vet. J., vol. 59, p. 16; Pereira et al., 2011, Vet. Microbiol., vol. 148, p. 117)

Next to the acquired immune system, humans and most animals also have an innate immunity, which is available for immediate response to threats, by activation of type 1 interferons and pro-inflammatory cytokines such as: interleukin (IL-)1beta, IL6, IL8, IL12 and tumour necrosis factor alpha. As more became known of the innate immune system, initial assumptions that this was a simple or primitive system, were soon set aside; the innate immune system turns out to be highly complex, with specific receptors and a multitude of factors with agonist or antagonist activity. Also, the primary innate immune response is the indispensable basis for the secondary acquired immune response

Central to the innate immune response is the recognition of conserved molecular signatures from pathogens, by pattern recognition receptors (PRR). An important group of such PRRs are the so-called Toll-like receptors (TLR). TLRs have evolved to recognize highly conserved structures of viral (TLR 3, 7, 8, and 9) and bacterial (TLR1, 2, 4, 5, 6, 7, and 9) origin. This specificity allows TLRs to rapidly detect the presence of an invading micro-organism and subsequently initiate inflammatory and antimicrobial immune responses. In addition, TLRs expressed on dendritic cells and B-lymphocytes initiate antigen-specific adaptive immune responses in the secondary immune response. (Botos et al., 2011, Structure, vol. 19, p. 447; Jin & Lee, 2008, Immunity, vol. 29, p. 182).

Ligands for TLRs range from bacterial lipoproteins (TLR2), lipopolysaccharide (TLR4) and flagellin (TLR5) to bacterial CpG-rich DNA (TLR9) and double stranded RNA (TLR3) or single stranded RNA (TLR7 and 8). TLRs are type I transmembrane glycoproteins characterized by an extracellular leucine-rich repeat domain and an intracellular Toll/IL-1 receptor domain. Most TLRs use MyD88 as a universal adapter protein via a cascade of intracellular signalling to activate the transcription factor NFkB. The activation of TLRs is the ligand-induced dimerisation of a TLR; the subsequent interaction of the two TIR domains is the event that initiates the recruitment of MyD88 and IRAK proteins. The TLR-dimers can be heterodimers of different TLRs, this is considered to contribute to broadening of the receptors' repertoire.

For example TLR2 heterodimers recognise bacterial lipoproteins such as the diacylated lipoproteins from Gram-positive bacteria by a TLR 2-TLR 6 heterodimer, and triacylated lipoproteins from Gram-negative bacteria by TLR 1-TLR 2 heterodimer. TLR2 homodimers can recognise the artificial lipopeptide Pam2Cys. TLR1/2 uses CD14 as co-factor, and TLR2/6 uses CD36 as cofactor. (Jin et al., 2008, supra).

TLR 2 is classified as CD282, and is expressed on the surface of a variety of immune cells such as neutrophils, macrophages and dendrocytes. TLR2 is involved in the process leading to Gram-positive shock syndrome, as this could be prevented by an antibody (T2.5) that bound to TLR2 and inhibited its activation (Meng et al., 2004, The J. of Clin. Invest., vol. 113, p. 1473). Among many other functions, TLR2 is involved in the innate immunity to S. aureus. This was demonstrated in different ways: S. aureus bacterial infection increased in number and severity both in TLR2 knockout mice infected with wildtype S. aureus, and in normal mice infected with an S. aureus strain defective in lipoprotein production. (Schmaler et al., 2010, Int. J. of Med. Microbiol., vol. 300, p. 155). Most studies on the structure and function of TLRs have been done with cells from human and mouse origin. The structures of TLRs in other mammals have been found to be highly conserved. In birds, some differences to the TLR system were found. However TLR2 structure and function was mainly conserved (Brownlie & Allan, 2011, Cell Tissue Res., vol. 343, p. 121). Interestingly, in chickens one TLR2 heterodimer combined the functions of TLR1/2 and TLR2/6 of mammals: the chicken TLR2type2/TLR16 heterodimer was capable of binding both diacylated and triacylated peptides (Keestra et al. 2007, The J. of Immunol., vol. 178, p. 7110).

In the nucleotide databases a wide variety of TLR2 nucleotide sequences are available, both from humans and from a wide variety of animals: mouse and several species of rodents, chimpanzee, bovines, goat, sheep, antelope, dog, horse, swine, chicken, several species of fish, etc.

Staphylococci can be non-pathogenic such as S. canosus. In evolution some Staphylococci (such as S. aureus) have acquired a large amount of additional genetic elements that allow it to express virulence factors. This makes the genome of S. aureus considerably larger (up to 2.9 Mb) than that of non-pathogenic species (commonly 2.3-2.5 Mb). These mobile genomic elements that encode virulence factors are so called pathogenicity islands; for S. aureus: SaPI. (Feng et al., 2008, FEMS Microbiol. Rev., vol. 32, p. 23).

S. aureus has several SaPIs and can therefore express a wide arsenal of virulence factors; these include: adhesins, stress factors, and exoproteins. The exoproteins are enzymes, toxins and immunomodulators. The toxins include the well known toxic-shock syndrome toxin, which is a ‘superantigen’. Such superantigens are able to activate subsets of T-lymphocytes without antigenic specificity by interacting directly with MHC class II molecules on macrophage's and with the Vb chain of T-cell receptors. This causes a cytokine release leading to major systemic shock effects.

The immunomodulators that S. aureus secretes in different stages of infection assist the establishment and expansion of the bacterial infection; they reduce or evade the detection and the clearance of S. aureus by the immune- or the complement system, and the mobilisation of phagocytes, such as neutrophils, monocytes and macrophages. Some are for example: the chemotaxis inhibitory protein (CHIPS), the Staphylococcal complement inhibitor (SCIN), and the formyl peptide receptor-like 1 inhibitory protein (FLIPr). (Veldkamp & van Strijp, 2009, Adv. Exp. Med. Biol., vol. 666, p. 19).

A group of 14 genes has been identified that potentially encode proteins that resemble superantigens, but they lack the MHC binding capacity. Hence their name: staphylococcal superantigen-like (SSL) proteins. Previously these proteins were known as staphylococcal exotoxin-like (SET) proteins (Arcus et al., 2002, J. of Biol. Chem., vol. 277, p. 32274), but nomenclature was disorderly for SETs from various S. aureus strains. These have now been renamed to SSL 1-14 (Lina et al., 2004, J. of Infect. Dis., vol. 189, p. 2334), whereby the SSL proteins are named in the order in which their encoding gene occurs on the S. aureus genome. (Smyth et al., 2007, J. of Med. Microbiol., vol. 56, p. 418). SSL1-11 are on SaPI2 (previously named: vSa alpha), and 12-14 on cluster IEC-2 of the S. aureus genome. Not every SSL gene is present in every S. aureus isolate, and alternatively, for some SSL genes there exist some allelic variants.

SSLs are polymorphic paralogs of the superantigens, which have elements of sequence and structure in common. However the few SSLs that have been characterised, were found to each have very different functions: SSL5 binds to P-selectin glycoprotein ligand1 (PSGL1) on neutrophils, thereby blocking their mobilisation to a site of infection; SSL7 binds to human IgA and to complement factor C5; SSL10 inhibits CXCR4; and SSL11 binds to the myeloid receptor FcαRI (CD 89). (Fraser & Proft, 2008, 1 mm. Reviews, vol. 225, p. 226; Bestebroer et al., 2009, Blood., vol. 113, p. 328; Walenkamp et al., 2009, Neoplasia, vol. 11, p. 333; Langley et al., 2010, Crit. Rev. in Immunol., vol. 30, p. 149).

Based on these findings the SSL proteins have been suggested to be immune evasion proteins, but most SSLs have thus not yet been studied or characterised. Many SSL gene- and putative protein sequences are available in databases such as NCBI's GenBank™, but such publications are merely based on in silico analyses of S. aureus genomic data. Recently the regulation of SSL gene expression was analysed (Benson et al., 2011, Molec. Microbiol., vol. 81, p. 659). SSLs have been described for use in targeting of a chosen antigen to antigen-presenting cells (WO 2005/092918), although only the use of SSL7 and 9 was disclosed in detail.

The many SSL sequences published, are derived from S. aureus isolates from humans but also from a variety of animal species: cow, goat, sheep, rabbit, and chicken (Smyth et al., 2007, supra).

The major problem with S. aureus developing today is the increased occurrence of strains that are resistant against multiple antibiotics, mostly indicated as methicillin resistant Staphylococcus aureus (MRSA). When these infect a carrier, there are few options left for treatment. One cause of preventive action is the reduction of the general use of antibiotics, in humans, but particularly in animals; an alternative is the search for an effective vaccine.

The major principle of clearing an S. aureus infection is phagocytosis, followed by intracellular killing by phagocytes. This is most effective after opsonisation of the bacterium by antibodies and fixation by complement. Therefore, any vaccine directed against the bacterium must induce sufficient antibody levels in a subject, either systemically, or locally to enable opsonisation. This has not yet been generally successful; for years many possible candidate antigens for S. aureus vaccines have been investigated either from the bacteria's complex outer surface, or from the great many molecules the bacterium excretes in the various phases of its lifecycle. Few antigens have shown promising results, and no generally effective vaccine is commercially available. (Broughan et al., 2011, Exp. Rev. Vacc., vol. 10, p. 695; Thomsen et al., 2010, Human Vacc., vol. 12, p. 1068).

Consequently, until today, and in spite of great potential advantages and many attempts over time, there is no effective vaccine against S. aureus for humans and animals.

It is an object of the present invention to accommodate to this urgent need in the field, and to provide an effective vaccine against Staphylococcus aureus for use in humans and animals.

It was surprisingly found that this object could be met through the use of a Staphylococcal superantigen-like 3 (SSL3) protein, or a homolog of said SSL3 protein, or an immunogenic fragment of either protein, in a vaccine against S. aureus.

The crucial discovery made by the inventors was the finding that SSL3 binds to the extracellular domain of TLR2, and potently inhibits the activation of TLR2 and thereby its capability to initiate an innate immune response. SSL4 was found to have the same inhibitory effect on TLR2, albeit to a lesser extent; as SSL4 is highly identical to SSL3, it is considered a homolog of SSL3. The inhibition of TLR2 by SSL3, or by a homolog was also possible by using a fragment of either of the two proteins, comprising the C-terminal part of SSL3, or of the homolog.

Although they do not wish to be bound by theory, the inventors suggest that when S. aureus expresses and secretes SSL3 and SSL4 upon infection of a host, these proteins inhibit the normal activation of TLR2. This provides a blockade of the innate immune response that would otherwise occur when the native TLR2 would recognise lipoproteins from S. aureus, and would initiate the production of cytokines, and the mobilisation of phagocytes. This provides S. aureus with a clear path to establish its infection undisturbed, and create tolerance once infection is established.

The advantageous utility of this discovery is in the use of SSL3, SSL4, or a fragment of either of these proteins, as a subunit vaccine against S. aureus. This way, by the vaccination of a target human or animal, the vaccinee will generate specific antibodies against the SSL3 or SSL4 proteins, or their fragments. These antibodies will inactivate the SSL3 and SSL4 secreted by the infecting S. aureus, and this will prevent the inhibitory effect these SSL proteins would otherwise have on TLR2. Thereby restoring the capability of the innate immune system to act at its full strength, and allowing the immune system to proceed with an effective clearance of the infecting S. aureus bacteria. When put in a popular way: the vaccination will ‘inhibit the inhibitor’.

This has several advantages over previous vaccination approaches: because no opsonisation of S. aureus is required, the antibody titers that need to be reached by the vaccine according to the invention do not need to be very high. On the other hand, as is disclosed herein, SSL3 and SSL4 were found to be highly immunogenic, as most healthy humans and animals tested already possessed clearly detectable antibody levels against these proteins. As a result, a vaccination with SSL3, or its homologs, or fragments of either, will for most vaccinees be a booster vaccination, leading to enhanced antibody titers.

This was not at all straightforward: even though TLR2 is an important factor in the innate immunity, there was no indication in the prior art that any one of the many exoproteins of S. aureus would interact with this receptor, let alone inhibit its activation directly. Also, it was in no way evident that an SSL protein could interact with a TLR receptor, as the SSL proteins of which the function was known, all have very different activities; indeed: of the SSL1-11, none of the others was found to have any (similar) activity towards TLR2.

Petzl et al. (2008, supra), and Yang et al. (2008, Molec. Immunol., vol. 45, p. 1385), have speculated on the role of TLR2 and TLR4 in subclinical S. aureus infection in bovine mastitis. However, their working hypothesis presumed an increase of TLR2 abundance after S. aureus infection, and no molecular mechanism could be found to explain why NFkB levels did not increase. They considered that S. aureus posed a paradox.

SSL3 and SSL4 are the first non-antibody proteins that are now known to inhibit the activation of TLR2 by directly binding to it, in a molecular interaction; the only other protein of which a similar binding and inhibition of activation of TLR2 is known, is the T2.5 antibody (Meng et al., 2004, supra). In the prior art other proteins and factors have been described that bind TLR2 and inhibit its functioning. However, these actually inhibit the factors ‘downstream’ of TLR2 in the signalling cascade of the innate immune system, not the activation of TLR2 itself. For example:

Pathak et al. (2007, Nature Immunol., vol. 8, p. 610), described a direct interaction between the early secreted antigen ESAT-6 of Mycobacterium tuberculosis and TLR2. However, the binding of ESAT-6 to the extracellular domain of TLR2 activated the intracellular signalling molecule Akt and this prevented the interaction between the adaptor MyD88 and its downstream kinase IRAK4, which both are active downstream of TLR2 activation. Therefore, ESAT-6 inhibited the signalling by TLR2 once it was activated, not the activation of TLR2 itself.

Similarly, the small molecule compound E567 is an inhibitor of the signalling by (activated) TLR2, not of the activation of TLR2 per se; E567 targets the adapter proteins MyD88 and MyD88 adapter-like, which are both involved in the signalling pathways downstream in the cascade of TLR2 and TLR4 (Zhou et al., 2010, Antiviral Res., vol. 87, p. 295).

Therefore in one aspect the invention relates to a Staphylococcal superantigen-like 3 (SSL3) protein, or a homolog of said SSL3 protein, or an immunogenic fragment of either protein, for use in a vaccine against Staphylococcus aureus.

According to the prior art, an “SSL3 protein” is a protein that is encoded by the gene on the genome of S. aureus that is named SSL3, because of its relative location in the order of SSL genes (Smyth, 2007, supra). In addition, an SSL3 protein for the invention has the characterising feature that it is capable of direct binding to TLR2, and thereby inhibiting the activation of the TIR domain of said TLR2 by a TLR2 ligand such as a bacterial lipoprotein. Methods to determine such binding, and such inhibition are described and exemplified in detail herein.

The amino acid sequence of a reference SSL3 protein for use according to the invention, is SSL3 from S. aureus strain NCTC 8325, and is represented as SEQ ID NO: 1. Examples of further SSL3 proteins for use according to the invention are displayed in Table 1. This displays the details of a representative number of SSL3 proteins from S. aureus strains, from humans and animals, and from regular S. aureus strains, or MRSA type strains. Most of these are derived from a public database, with the exception of a number of SSL3 proteins from bovine isolates of S. aureus, that were analysed in house. Their amino acid sequences are presented in SEQ ID NO's: 2-5.

The SSL 3 proteins for use according to the invention, that are listed in Table 1 were compared by multiple amino acid sequence alignment, a picture of a specific grouping emerged: amongst them the SSL3 protein were very conserved, and none had an amino acid sequence identity to any of the others, or to the reference SSL3 protein sequence (SEQ ID NO: 1), that was less than 90%; Table 2 presents the % identity of the mutual alignment results for SSL3 proteins, and FIG. 9, presents these results in a dendrographic tree.

Therefore, in a preferred embodiment the invention relates to the SSL3 protein for use according to the invention, wherein the SSL3 protein is a protein comprising an amino acid sequence having at least 90% amino acid sequence identity to the amino acid sequence of SEQ ID NO. 1.

This definition of SSL3 proteins for use according to the invention by the minimal level of amino acid sequence identity, in addition with the requirement for TLR2 inhibition as described, sets the said SSL3 proteins clearly apart from any protein in the prior art; the best match of SEQ ID NO: 1 to any other amino acid sequences of unrelated proteins in the public databases was 55% identity or less; whereby an ‘unrelated’ protein is one of which the annotation indicated it was not an SSL3 or an SSL4 protein.

This also applies to the other SSL proteins from S. aureus; an example is presented in Table 5, and is described below.

In a preferred embodiment, the SSL3 protein for use according to the invention, has at least 91% amino acid sequence identity to the amino acid sequence of SEQ ID NO. 1, more preferably, 92, 93, 94, 95, 96, 97, 98, 99, or even 100% sequence identity to the amino acid sequence of SEQ ID NO. 1, in that order of preference.

For the invention, the term “comprising” (as well as variations such as “comprise”, “comprises”, and “comprised”) as used herein, refer(s) to all elements, and in any possible combination conceivable for the invention, that are covered by or included in the text section, paragraph, claim, etc., in which this term is used, even if such elements or combinations are not explicitly recited; and not to the exclusion of any of such element(s) or combinations. Consequently, any such text section, paragraph, claim, etc., can also relate to one or more embodiment(s) wherein the term “comprising” (or its variants) is replaced by terms such as “consist of”, “consisting of”, or “consist essentially of”.

TABLE 1
List of SSL3 and SSL4 amino acid sequences, used for the multiple alignments

| | Isolated | | | | |
|---|---|---|---|---|---|
| Strain | Species | Country | Remarks | SSL3 Acc. no. | SSL4 Acc. no. |
| RF122 | Bovine | Ireland | mastitis | YP_415879 | — |
| JH9 | Human | USA | MRSA/VISA | YP_001245828 | — |
| Mu50 | Human | Japan | MRSA | NP_370948 | — |
| N315 | Human | Japan | MRSA | — | NP_373635 |
| COL | Human | England | | YP_185360 | YP_185362 |
| MW2 | Human | USA | CA-MRSA | NP_645201 | NP_645202 |
| CF-Marseille | Human | France | MRSA | ZP_04839712 | — |
| TCH130 | Human | USA | MRSA | ZP_04869322 | — |
| 55/2053 | Human | England | | — | ZP_05600974 |
| A9635 | Human | USA | MRSA | — | ZP_05687415 |
| | | | | | ZP_05687416 |
| A9299 | Human | USA | MRSA | | ZP_05689242 |
| A6300 | Human | USA | MRSA | ZP_05693238 | — |
| C160 | Human | England | BIGSP⁽¹⁾ | — | ZP_06310906 |
| D139 | Human | England | BIGSP | — | ZP_06323515 |
| A9754 | Human | USA | BIGSP | ZP_06790788 | — |
| ST398 | Human | Netherlands | MRSA | — | CAQ48930 |
| | | | | | CAQ48931 |
| ED133 | Ovine | France | mastitis | ADI96978 | ADI96980 |
| JKD6159 | Human | Australia | MRSA | — | ADL22333 |
| | | | | | ADL22332 |
| JKD6009 | Human | Australia | MRSA/VSSA | ZP_03565895 | — |
| CGS03 | Human | USA | | — | EFT86040 |
| O11 | Ovine | France | mastitis | EGA96996 | — |
| O46 | Ovine | France | mastitis | — | EGB00138 |
| 21193 | Human | | Craig Venter Inst.⁽²⁾ | EGG68742 | EGG68638 |
| 21310 | Human | | Craig Venter Inst. | — | EGL91296 |
| 21235 | Human | | Craig Venter Inst. | EGS83188 | EGS83190 |
| 21266 | Human | | Craig Venter Inst. | EGS84045 | EGS84008 |
| 21269 | Human | | Craig Venter Inst. | EGS84524 | EGS84548 |
| 21259 | Human | | Craig Venter Inst. | — | EGS89332 |
| LGA251 | Bovine | UK | | CCC87131 | CCC87132 |
| MSSA476 | Human | UK | MSSA | YP_042511 | — |
| MRSA 252 | Human | UK | EMRSA | — | YP_039876 |
| | | | | | YP_039877 |
| NCTC8325 | Human | UK | non-MRSA | SEQ ID NO: 1 | SEQ ID NO: 6 |
| | | | | YP_498973 | YP_498975 |
| S1444 | Bovine | Germany | mastitis | SEQ ID NO: 2 | SEQ ID NO: 7 |
| S1446 | Bovine | Spain | mastitis | SEQ ID NO: 3 | SEQ ID NO: 8 |
| S1449 | Bovine | France | mastitis | SEQ ID NO: 4 | — |
| S1454 | Bovine | Canada | mastitis | SEQ ID NO: 5 | — |

⁽¹⁾Isolate sequenced by Broad Institute Sequencing Genomic Platform - no information available
⁽²⁾Isolate sequenced by Craig Venter Institute- no information available

TABLE 2
Multiple alignment scores for SSL3 proteins in % amino acid sequence identity

| | SSL3 from: | 1 | 2 | 3 | 4 | 5 | 6 | 7 | 8 | 9 | 10 | 11 | 12 | 13 | 14 | 15 | 16 | 17 | 18 | 19 | 20 | 21 | 22 |
|---|---|---|---|---|---|---|---|---|---|---|---|---|---|---|---|---|---|---|---|---|---|---|---|
| 1 | NCTC8325¹⁾ | | | | | | | | | | | | | | | | | | | | | | |
| 2 | MW2²⁾ | 99 | | | | | | | | | | | | | | | | | | | | | |
| 3 | MSSA476 | 99 | 99 | | | | | | | | | | | | | | | | | | | | |
| 4 | JKD6009 | 100 | 99 | 99 | | | | | | | | | | | | | | | | | | | |
| 5 | 21235 | 91 | 91 | 91 | 92 | | | | | | | | | | | | | | | | | | |
| 6 | 21269 | 91 | 91 | 91 | 91 | 93 | | | | | | | | | | | | | | | | | |
| 7 | O11³⁾ | 91 | 91 | 90 | 91 | 93 | 99 | | | | | | | | | | | | | | | | |
| 8 | LGA251 | 91 | 91 | 90 | 91 | 99 | 99 | 92 | | | | | | | | | | | | | | | |
| 9 | A9754 | 99 | 98 | 97 | 100 | 91 | 91 | 90 | 90 | | | | | | | | | | | | | | |
| 10 | Mu50⁴⁾ | 97 | 97 | 97 | 98 | 92 | 92 | 91 | 92 | 97 | | | | | | | | | | | | | |
| 11 | COL⁵⁾ | 99 | 98 | 97 | 100 | 91 | 90 | 90 | 90 | 100 | 97 | | | | | | | | | | | | |
| 12 | JH9⁶⁾ | 97 | 97 | 96 | 98 | 92 | 91 | 91 | 91 | 96 | 99 | 96 | | | | | | | | | | | |
| 13 | CF-Marseille | 97 | 97 | 96 | 98 | 92 | 91 | 91 | 91 | 96 | 99 | 96 | 99 | | | | | | | | | | |
| 14 | 21193⁷⁾ | 96 | 95 | 95 | 96 | 91 | 91 | 91 | 91 | 95 | 96 | 95 | 95 | 95 | | | | | | | | | |
| 15 | TCH130 | 90 | 90 | 90 | 90 | 95 | 94 | 93 | 95 | 89 | 91 | 90 | 91 | 91 | 91 | | | | | | | | |
| 16 | ED133 | 95 | 94 | 94 | 96 | 93 | 92 | 91 | 92 | 94 | 96 | 94 | 96 | 96 | 95 | 91 | | | | | | | |
| 17 | A6300 | 97 | 96 | 97 | 97 | 92 | 91 | 90 | 91 | 96 | 99 | 96 | 99 | 99 | 95 | 91 | 96 | | | | | | |
| 18 | 21266 | 96 | 94 | 94 | 96 | 92 | 91 | 91 | 92 | 95 | 96 | 95 | 96 | 96 | 95 | 91 | 96 | 95 | | | | | |
| 19 | RF122 | 95 | 94 | 93 | 95 | 91 | 91 | 91 | 90 | 94 | 95 | 94 | 95 | 95 | 94 | 90 | 95 | 94 | 94 | | | | |
| 20 | S1444 | 92 | 92 | 92 | 92 | 99 | 94 | 94 | 98 | 91 | 91 | 91 | 92 | 92 | 92 | 95 | 93 | 92 | 92 | 92 | | | |
| 21 | S1446 | 95 | 94 | 93 | 96 | 91 | 91 | 91 | 90 | 94 | 95 | 94 | 95 | 95 | 94 | 90 | 95 | 96 | 95 | 94 | 91 | | |
| 22 | S1449 | 95 | 94 | 94 | 96 | 91 | 91 | 91 | 91 | 94 | 95 | 94 | 95 | 95 | 94 | 90 | 95 | 94 | 94 | 100 | 91 | 91 | |
| 23 | S1454 | 95 | 94 | 94 | 96 | 91 | 91 | 91 | 90 | 94 | 95 | 94 | 95 | 95 | 94 | 90 | 95 | 94 | 96 | 94 | 91 | 99 | 93 |

SSL3 sequences representative for others:
¹⁾NCTC8325 for 21189
²⁾MW2 for ATCC51811 and TCH70
³⁾O11 for O46
⁴⁾Mu50 for N315, Mu3, A9763, A9299, A8115, ED98, A8117, ECT-R2 and 21318
⁵⁾COL for FPR-3757, Newman, TCH1516, 132, ATCC BAA-39, TW20, JKD6008, CGS01, MRSA131 and TO131
⁶⁾JH9 for JH1, A9717, A6224, A5937, A10102, A8819, A8796, CGS03 and 21172
⁷⁾31193 for 21305

TABLE 3
Multiple alignment scores for SSL4 proteins in % amino acid sequence identity

| | SSL4 from: | 1 | 3 | 4 | 5 | 6 | 7 | 8 | 9 | 11 | 12 | 13 | 14 | 15 | 16 | 17 | 18 | 19 | 20 | 21 | 22 | 23 | 24 | 25 | 26 | 27 | 28 | 29 |
|---|---|---|---|---|---|---|---|---|---|---|---|---|---|---|---|---|---|---|---|---|---|---|---|---|---|---|---|---|
| 1 | 21193 | | | | | | | | | | | | | | | | | | | | | | | | | | | |
| 3 | O46 | 92 | | | | | | | | | | | | | | | | | | | | | | | | | | |
| 4 | MW2 | 84 | 84 | | | | | | | | | | | | | | | | | | | | | | | | | |
| 5 | 21259 | 86 | 85 | 85 | | | | | | | | | | | | | | | | | | | | | | | | |
| 6 | NCTC 8325 | 86 | 84 | 85 | 97 | | | | | | | | | | | | | | | | | | | | | | | |
| 7 | 21269 | 92 | 98 | 82 | 86 | 82 | | | | | | | | | | | | | | | | | | | | | | |
| 8 | COL | 85 | 82 | 84 | 96 | 98 | 82 | | | | | | | | | | | | | | | | | | | | | |
| 9 | 21266 | 87 | 84 | 85 | 98 | 98 | 84 | 97 | | | | | | | | | | | | | | | | | | | | |
| 11 | 21235 | 72 | 95 | 83 | 79 | 80 | 96 | 78 | 80 | | | | | | | | | | | | | | | | | | | |
| 12 | LGA251 | 71 | 72 | 80 | 82 | 79 | 73 | 78 | 80 | 95 | | | | | | | | | | | | | | | | | | |
| 13 | ED133 | 72 | 95 | 84 | 80 | 79 | 94 | 78 | 80 | 95 | 93 | | | | | | | | | | | | | | | | | |
| 14 | N315 | 88 | 84 | 86 | 88 | 89 | 89 | 87 | 90 | 79 | 79 | 91 | | | | | | | | | | | | | | | | |
| 15 | CGS03 | 88 | 88 | 76 | 80 | 80 | 85 | 78 | 80 | 95 | 94 | 95 | 90 | | | | | | | | | | | | | | | |
| 16 | A9299 | 84 | 83 | 92 | 72 | 73 | 80 | 71 | 75 | 91 | 91 | 92 | 84 | 94 | | | | | | | | | | | | | | |
| 17 | JKD6159a | 88 | 79 | 80 | 77 | 78 | 80 | 76 | 78 | 73 | 74 | 76 | 83 | 78 | 90 | | | | | | | | | | | | | |
| 18 | MRSA252a | 63 | 63 | 65 | 61 | 62 | 62 | 63 | 62 | 63 | 64 | 65 | 64 | 77 | 81 | 68 | | | | | | | | | | | | |
| 19 | A9635a | 70 | 62 | 63 | 63 | 63 | 64 | 63 | 65 | 65 | 67 | 65 | 65 | 78 | 83 | 69 | 92 | | | | | | | | | | | |
| 20 | ST398a | 62 | 63 | 64 | 63 | 64 | 62 | 62 | 64 | 64 | 66 | 63 | 64 | 61 | 84 | 66 | 92 | 94 | | | | | | | | | | |
| 21 | JKD6159b | 71 | 70 | 66 | 67 | 67 | 71 | 65 | 67 | 60 | 59 | 60 | 69 | 68 | 67 | 69 | 60 | 60 | 60 | | | | | | | | | |
| 22 | D139 | 59 | 59 | 63 | 60 | 60 | 59 | 59 | 61 | 60 | 61 | 60 | 60 | 57 | 74 | 63 | 80 | 80 | 80 | 67 | | | | | | | | |
| 23 | 21310 | 59 | 60 | 62 | 60 | 59 | 60 | 58 | 59 | 60 | 61 | 60 | 61 | 68 | 75 | 64 | 82 | 82 | 84 | 68 | 89 | | | | | | | |
| 24 | ST398b | 62 | 62 | 64 | 62 | 63 | 62 | 62 | 63 | 60 | 59 | 60 | 59 | 61 | 59 | 67 | 69 | 73 | 71 | 73 | 81 | 80 | | | | | | |
| 25 | C160b | 62 | 62 | 65 | 63 | 64 | 62 | 63 | 64 | 60 | 60 | 60 | 63 | 60 | 75 | 61 | 65 | 92 | 67 | 71 | 74 | 78 | 76 | | | | | |
| 26 | MRSA252b | 65 | 64 | 64 | 63 | 64 | 62 | 61 | 62 | 59 | 60 | 59 | 65 | 63 | 61 | 60 | 65 | 67 | 68 | 74 | 71 | 77 | 80 | 90 | | | | |
| 27 | 55/2053b | 66 | 63 | 65 | 65 | 66 | 66 | 65 | 66 | 59 | 60 | 60 | 67 | 64 | 61 | 61 | 66 | 68 | 69 | 75 | 77 | 78 | 78 | 93 | 98 | | | |
| 28 | A9635b | 62 | 70 | 64 | 66 | 66 | 70 | 64 | 66 | 59 | 58 | 57 | 68 | 68 | 66 | 65 | 63 | 62 | 62 | 79 | 71 | 70 | 82 | 81 | 83 | 86 | | |
| 29 | S1444 | 87 | 91 | 88 | 88 | 86 | 92 | 87 | 89 | 80 | 79 | 80 | 91 | 85 | 79 | 83 | 64 | 63 | 64 | 68 | 61 | 62 | 64 | 64 | 67 | 68 | 68 | |
| 30 | S1446 | 94 | 90 | 89 | 87 | 87 | 91 | 86 | 88 | 77 | 75 | 77 | 88 | 83 | 76 | 79 | 62 | 63 | 63 | 69 | 59 | 60 | 64 | 63 | 64 | 62 | 68 | 92 |

Therefore, in a more preferred embodiment, the SSL3 protein for use according to the invention consists of the amino acid sequence of any one SEQ ID NO. selected from the group consisting of SEQ ID NO. 1 through SEQ ID NO: 5.

For the invention, the term “protein” refers to any molecular chain of amino acids. A protein is not necessarily of a specific length, structure or shape and can, if required, be modified in vivo or in vitro, by, e.g. glycosylation, amidation, carboxylation, phosphorylation, pegylation, or changes in spatial folding. The protein can be a native or a mature protein, a pre- or pro-protein, or a functional fragment of a protein. A protein can be of biologic or of synthetic origin, and may be obtained by isolation, purification, assembly etc. A protein may be a chimeric- or fusion protein, created from fusion by biologic or chemical processes, of two or more proteins protein fragments. Inter alia, peptides, oligopeptides and polypeptides are included within the term protein.

A “homolog” for use according to the invention is a protein that is homologous to, and has the essential characteristics of, an SSL3 protein for use according to the invention. In particular this regards being capable of direct binding to TLR2 and thereby inhibit the activation of the TIR domain of said TLR2 by a TLR2 ligand such as a bacterial lipoprotein.

As described above, no unrelated protein had more than 55% amino acid sequence identity to the SSL3 protein for use according to the invention.

Therefore, in a preferred embodiment, the homolog for use according to the invention, is a protein that is capable of direct binding to TLR2 and thereby inhibit the activation of the TIR domain of said TLR2 by a TLR2 ligand such as a bacterial lipoprotein, and wherein said protein comprises an amino acid sequence having at least 56% amino acid sequence identity to the amino acid sequence of SEQ ID NO. 1.

“Direct binding” for the invention has been described above, and involves a direct molecular interaction, without intermediate molecules being involved.

More preferably, the homolog for use according to the invention has at least 60% amino acid sequence identity with SEQ ID NO: 1, even more preferably 65, 70, 75, 80, 85, 86, 87, 88, or even 89% sequence identity to the amino acid sequence of SEQ ID NO. 1, in that order of preference.

The inventors noted that in SaPI2 on the genome of S. aureus bacteria isolated from some animal species, specifically bovine S. aureus isolates, no copy of an SSL3 gene was present, in stead there was a copy of an SSL4 gene. (Smyth et al., 2007, supra). When tested, the SSL4 proteins were found to share with SSL3 the capability for use according to the invention, only to a lesser extent. Therefore, the inventors propose that an SSL4 protein is a natural homolog for SSL3, and appears in a number of S. aureus strains.

The amino acid sequence of a reference SSL4 protein for use according to the invention, is SSL4 from S. aureus strain NCTC 8325, and is represented as SEQ ID NO: 6.

SEQ ID NO: 1 and SEQ ID NO: 6 have 62% amino acid sequence identity.

Examples of further SSL4 proteins for use according to the invention are displayed in Table 1. This displays the details of a representative number of SSL4 proteins from S. aureus strains, from humans and animals, and from regular S. aureus strains, or MRSA type strains. Most of these are derived from a public database, with the exception of a number of SSL4 proteins from bovine isolates of S. aureus, that were analysed in house. Their amino acid sequences are presented in SEQ ID NO's: 7-8.

The SSL4 proteins for use according to the invention, that are listed in Table 1 were compared by multiple amino acid sequence alignment. Table 3 presents the % identity of the mutual alignment results for SSL4 proteins, and FIG. 10, presents these results in a dendrographic tree.

Although quite well conserved amongst them, the SSL4 proteins were not so conserved as SSL3 proteins; their mutual amino acid sequence identity was between 57 and 98% (Table 3). Amino acid sequence identity with the reference SSL4 protein (SEQ ID NO: 6) was between 59 and 99%. The reason being that SSL 4 genes were found to appear in different allelic variants, named set2 and set9. This makes that the group of SSL4 proteins differs amongst themselves in length and in sequence.

Therefore in a further preferred embodiment, the homolog for use according to the invention is a protein, comprising an amino acid sequence having at least 59% amino acid sequence identity to the amino acid sequence of SEQ ID NO. 6.

The sequence identity to be calculated as described above, and over the full length of SEQ ID NO: 6.

More preferably, the homolog for use according to the invention has at least 60% amino acid sequence identity with SEQ ID NO: 6, even more preferably 62, 65, 70, 75, 80, 85, 90, 91, 92, 93, 94, 95, 96, 97, 98, or even 99% sequence identity to the amino acid sequence of SEQ ID NO. 6, in that order of preference.

In an even more preferred embodiment, the homolog for use according to the invention comprises the amino acid sequence of any one SEQ ID NO. selected from the group consisting of SEQ ID NO. 6 through SEQ ID NO: 8.

To compare SSL3 and SSL4 proteins, a number of representative of SSL3 and SSL4 proteins from the various subgroups seen in the dendrographic trees (FIGS. 9 and 10), were compared by multiple amino acid sequence alignment. This is presented in FIG. 11, as a textual output; Table 4 presents the corresponding amino acid sequence identity levels between SSL3 and SSL4 proteins, and correlates these to SEQ ID NO: 1 and 6.

This demonstrates that in spite of the variance in SSL4 proteins, the SSL3 and SSL4 are still within the definition of homologs of SSL3 for use according to the invention, which uses a cut off of more than 55% amino acid sequence identity to SEQ ID NO: 1.

When comparing SSL3 and SSL4 proteins in detail, it was apparent that SSL4 proteins are generally shorter, lacking a section of sequence in the N-terminal half as compared to SSL3. Nevertheless, the C-terminal halves of SSL3 and SSL4 were found to be highly conserved. The inventors therefore speculate that the active site of SSL3 and SSL4 for binding to TLR2 is in the C-terminal half of the proteins.

TABLE 4
Pairwise alignments of SSL3 and SSL4 proteins

| | Name | Accesion no. | 1 | 2 | 3 | 4 | 5 | 6 | 7 | 8 | 9 | 10 |
|---|---|---|---|---|---|---|---|---|---|---|---|---|
| 1 | 21193-SSL3 | EGG68742 | | 91 | 95 | 95 | 96 | 56 | 59 | 59 | 68 | 61 |
| 2 | LGA251-SSL3 | CCC87131 | 91 | | 90 | 90 | 91 | 56 | 58 | 57 | 66 | 60 |
| 3 | COL-SSL3 | YP_185360 | 95 | 90 | | 96 | 99 | 57 | 59 | 58 | 67 | 61 |
| 4 | A6300-SSL3 | ZP_05693238 | 95 | 91 | 96 | | 97 | 56 | 60 | 58 | 67 | 61 |
| 5 | NCTC 8325-SSL3 (1) | YP_498973 | 96 | 91 | 99 | 97 | | 57 | 60 | 59 | 68 | 62 |
| 6 | s1444-SSL4 | SEQ ID NO: 7 | 56 | 56 | 56 | 56 | 57 | | 87 | 64 | 61 | 86 |
| 7 | COL-SSL4 | YP_185362 | 59 | 58 | 56 | 56 | 57 | 87 | | 62 | 59 | 98 |
| 8 | ST398-SSL4 | CAQ48930 | 56 | 58 | 56 | 56 | 57 | 65 | 62 | | 80 | 64 |
| 9 | D139-SSL4 | ZP_06323515 | 67 | 66 | 66 | 66 | 67 | 62 | 59 | 80 | | 60 |
| 10 | NCTC 8325-SSL4 (2) | YP_498975 | 61 | 60 | 58 | 58 | 59 | 86 | 98 | 64 | 60 | |

(1) SEQ ID NO: 1
(2) SEQ ID NO: 6

A “fragment” for use according to the invention is a protein which is a part of either an SSL3 protein for use according to the invention, or a part of a homolog for use according to the invention. Said protein fragment for the invention still has the capacity to bind directly to TLR2 and thereby inhibit the activation of the TIR domain of said TLR2 by a TLR ligand such as a bacterial lipoprotein.

A test for determining whether a particular fragment is a fragment for use according to the invention, can for example be performed using TLR2 expressing cells, as exemplified herein. When using primary cells of the immune system, the read-out usually employs IL8 production or NFkB expression. When used on recombinant cells expressing a heterologous TLR2, often the expression of a reporter gene is used. Such a system can indicate the activation of TLR2 by a TLR2 ligand such as a bacterial lipoprotein for example by detection of a reduction in luciferase or GFP expression as compared to uninhibited TLR2 expressing cells. In this type of assay a fragment for use according to the invention can block the expression of such a reporter gene, so that inhibition of TLR2 is detected routinely.

The fragment for use according to the invention preferably achieves at least 50% inhibition of the activation of the TIR domain of TLR2 by a TLR2 ligand such as a bacterial lipoprotein, compared to an uninhibited culture. More preferably, 60, 70, 80, 90, or even 100% inhibition, in this order of preference.

Bacterial lipoproteins for use in such a test are commonly known and available; conveniently synthetic peptides are used such as: Pam2Cys, Pam3Cys, or MALP-2.

A fragment for use according to the invention can for example be a mature or processed form of an SSL3 protein or of a homolog for use according to the invention, i.e. without a ‘leader’, ‘anchor’, ‘signal’ or ‘tail’ sequence.

In a preferred embodiment, a fragment for use according to the invention is a part of a SSL3 protein, or of a homolog, both for use according to the invention, which comprises the C-terminal region of said SSL3 protein or homolog. This region was found to contain the TLR2 binding activity.

Examples of fragments for use according to the invention are: the region from amino acid numbers 127 to 326 of SEQ ID NO: 1, or the region from amino acids 79-278 of SEQ ID NO: 6, both 200 amino acids in length.

In a more preferred embodiment, a fragment for use according to the invention is a protein that is at least 200 amino acids in length, whereby the protein is a fragment taken from the C-terminal side from an SSL3 protein for use according to the invention, or from the C-terminal side from a homolog for use according to the invention. More preferably, said fragment is at least 175, 150, 100, 90, 80, 70, 60, or even 50 amino acids in length, taken from the C-terminal side of the SSL3 protein, or the homolog, both for use according to the invention.

The capability of such a preferred fragment to inhibit TLR2 activation, is demonstrated in FIG. 12: this compares the capacity to inhibit TLR2 activation by SSL3 and by a C-terminal fragment of SSL3, the amino acids 127-326 of SEQ ID NO: 1. Both are almost equally effective.

This is also established when comparing the C-terminal regions of SSL3 and SSL4 with the other SSL proteins of S. aureus; SSL 1, 2, and 5-14 are all about 200 amino acids in length. When aligning the amino acid sequences of the other SSLs (from S. aureus strain NCTC 8325) to the C-termini of SSL3 and of SSL4, the results show that although there is conservation, this does not exceed 46% amino acid sequence identity (for SSL11) to the C-terminal region of SSL3 (amino acid numbers 127-326 of SEQ ID NO: 1), see Table 5. Surprisingly the sequence identity between SSL3 and SSL4 in this region is 76%. Therefore the inventors speculate that this region holds the capability for inhibiting TLR2.

TABLE 5
List of pairwise alignments of the C-terminal ends of amino acid
sequences from SSL3 and SSL4 with different SSLs; all SSL
amino acid sequences are from S. aureus strain NCTC 8325.
pairwise alignment smade using Alignplus ™
(Scientific Educational Software), using default parameters.

| | | | % identity |
|---|---|---|---|
| protein | database acc. nr. | aa nrs. | to SSL3 |
| SSL3 | SEQ ID NO: 1 | 127-326 | 100 |
| | YP_498973 | | |
| SSL4 | SEQ ID NO: 6 | 79-278 | 76 |
| | YP_498975 | | |
| 1 | YP_498971 | 1-196 | 40 |
| 2 | YP_498972 | 1-201 | 44 |
| 5 | YP_498976 | 1-204 | 39 |
| 6 | YP_498978 | 1-201 | 44 |
| 7 | YP_498979 | 1-201 | 37 |
| 8 | YP_498980 | 1-202 | 40 |
| 9 | YP_498981 | 1-202 | 39 |
| 10 | YP_498982 | 1-197 | 31 |
| 11 | YP_498986 | 1-195 | 46 |
| 12 | YP_499668 | 1-205 | 25 |
| 13 | YP_499669 | 1-210 | 23 |
| 14 | YP_499670 | 1-209 | 23 |

A fragment for use according to the invention needs to be “immunogenic”, in order to have utility in a vaccine against S. aureus according to the invention. For the invention, the term ‘immunogenic’ refers to the capacity to induce a specific immune response that is effective in binding, inactivating, clearing, etc. of SSL3 or SSL4 protein from S. aureus. Such an immune response may be achieved by the induction of specific antibodies and/or by the generation of a cellular immune response, either of which should be able to interact with SSL3 or SSL4 as described.

As is well known in the art, proteins in order to be immunogenic need to be of a minimal length; typically 8-11 aa for MHC I receptor binding, and 11-15 aa for MHC II receptor binding (Germain & Margulies, 1993, Annu. Rev. Immunol., vol. 11, p. 403). Therefore an immunogenic fragment of an SSL3 protein or a homolog for use according to the invention, is at least 8 amino acids in length. More preferably a fragment for the invention is at least 10, 15, 20, 25, 50, 75, 100, 150 or 200 amino acids in length.

Immunogenic fragments, of which the immunogenicity still needs to be improved, can be presented to a target's immune system attached to, or in the context of, an immunogenic carrier molecule. Well known carriers are bacterial toxoids, such as Tetanus toxoid or Diphteria toxoid; alternatively KLH, BSA, or bacterial cell-wall components (derived from) lipid A, etc. may be used. Also polymers may be useful, or other particles or repeated structures such as virus like particles etc. The coupling of a fragment for use according to the invention to a carrier molecule can be done by methods known in the art, using chemical or physical techniques.

The determination of a whether a fragment for use according to the invention is immunogenic can be performed in several ways, well known in the art, using in vivo or in vitro models to test for a specific immune response. For example by generating tryptic digests of an SSL3 protein or a homolog for use according to the invention, testing the immunogenicity of the fragments obtained, and analysing the fragments that perform as desired. Or the fragments can be synthesized and tested as in the well known PEPSCAN method (WO 84/003564; WO 86/006487; and Geysen et al., PNAS USA, 1984, vol. 81, p. 3998). Alternatively, immunogenically relevant areas can be predicted by using well known computer programs. An illustration of the effectiveness of using these methods was published by Margalit et al. (1987, J. of Immunol., vol. 138, p. 2213) who describe success rates of 75% in the prediction of T-cell epitopes.

“Staphylococcus aureus” and ‘S. aureus’ for the invention are terms used to refer to the bacterial organism that is currently known by this name. However, in respect of the precise taxonomic classification of S. aureus, the skilled person will realise this may change over time as new insights can lead to reclassification into new or other taxonomic groups. However, as this does not change the characteristics or the protein repertoire of the organism involved, only its classification, such re-classified organisms are considered to be within the scope of the invention.

In that respect the invention intends to encompass all bacteria sub-classified from S. aureus for the invention, either as a sub-species, strain, isolate, genotype, serotype, variant or subtype and the like.

The SSL3 protein, the homolog, and the immunogenic fragment, all for use according to the invention, have an advantageous utility “for use in a vaccine against S. aureus”. As described above, such a vaccine would restore in a vaccinated human or animal the capacity of the innate immune system to attack and clear the infecting S. aureus bacteria. The vaccine can have any composition, and can take any form, which would be suitable for this purpose. Detailed embodiments of such a vaccine are described and exemplified herein.

An advantageous variation on a use for the invention as described above, is one wherein the vaccination of the human or animal target is not performed by a protein, such as an SSL3 protein, a homolog, or a fragment, all for use according to the invention; rather the vaccination would employ an antibody which is directed against such a protein. By the administration to a human or animal subject of such antibodies, these antibodies can immediately inactivate any SSL3 or SSL4 protein that might be present or circulating resulting from an active or emerging S. aureus infection.

The use of antibodies for vaccination is referred to as ‘passive vaccination’. This has a number of specific benefits over the use of active vaccination with antigenic proteins, mainly because of the speed of action: the antibodies are present and active in the human or animal target as soon as they have been administered, whereas an active immunisation with proteins may require up to two weeks to produce sufficient antibody titers.

An other advantage of passive vaccination is that this provides a therapy for those subjects, for which a classical immune response is not possible, or would not be effective enough; for example because of an immune-compromising condition or illness. Typically such targets are young, old, pregnant, or sick.

Therefore in a further aspect, the invention relates to an isolated antibody that can bind specifically to an SSL3 protein, or to a homolog of said SSL3 protein, or to an immunogenic fragment of either protein, for use in a vaccine against S. aureus.

The term “isolated” is to be interpreted as: isolated and/or purified from its natural environment, by deliberate action, and subsequently taken up into an appropriate composition or container.

An “antibody” is an immunoglobulin or an immunologically active part thereof, for instance a fragment that still comprises an antigen binding site, such as a (camelid) single chain antibody, a diabody, a domain antibody, bivalent antibody, or a Fab, Fab′, F(ab′)₂, Fv, scFv, dAb, or Fd fragment, or other antigen-binding subsequences of antibodies, all well known in the art.

For an antibody to “bind specifically” to a certain target, means that the antibody, or rather its antigen binding site(s), can engage in a molecular interaction with an epitope on an antigen, which interaction is so strong that it can be clearly differentiated from any non-specific, or transient binding; usually the differentiation is made by a dilution- or competition type immunological assay; for example an ELISA of immunofluorescence test.

It is common practice to define an antibody by its specificity, origination from the antigen to which the antibody was generated. Therefore the antibody for use according to the invention is identified by its specific antigen, an SSL3 protein, a homolog of said SSL3 protein, or an immunogenic fragment of either of these proteins, all for use according to the invention.

The antibody for use according to the invention can for example be generated in a healthy donor animal by classical vaccination, and purification from the donor's serum. For the present invention the donor animal would be vaccinated with an SSL3 protein, or a homolog, or a fragment, all for use according to the invention, or with any combination thereof. Typically some booster vaccinations would be given, to achieve very high antibody titers.

For some animals their use as donor of antibodies is already well known, for example: rabbit, and goat. Another example are chickens which can produce high levels of antibodies in the egg-yolk, so-called IgY. Preferably the donor animal is of the same species as the animal subject to be treated.

Alternatively, the antibody can be produced in vitro. One common way is via the well known monoclonal antibody technology from immortalized B-lymphocyte cultures (hybridoma cells), for which industrial scale production systems are known. Alternatively antibodies or fragments thereof may be expressed in any suitable recombinant expression system, through expression of the cloned Ig heavy- and/or light chain genes, in whole or in part. These can conveniently be purified and formulated to the desired form and quality. All this is well within the capabilities of the skilled person.

The production of antibodies by recombinant expression conveniently allows for adaptations to the antibody, for example to make it more stable, or more effective. For application to humans, but also for animal application, the recombinant methods allow the adaptation of the antibodies produced to make them resemble more the characteristics of the antibodies normal to that species. This way the antibodies are accepted better by the immune system of the human or animal target, preventing immunologic shock. Also this may considerably enhance the biological half-life of these antibodies in the target. Such adaptation is described as humanisation, bovinisation, caninisation, etc.

For the present invention the passive immunisation with an isolated antibody for use according to the invention, is advantageously applied to a human or animal target shortly before, during, or immediately following a surgical procedure. Such procedures are a well known cause of S. aureus infection. With these antibodies circulating at an adequate titre in a human or animal patient around the time of the surgical procedure, the possibility for an S. aureus which has infected tissues exposed during the procedure, to establish a productive infection can effectively be prevented.

Therefore in a preferred embodiment, the isolated antibody for use according to the invention, is applied to a human or animal subject prior to, during, or after a surgical procedure.

The skilled artisan is adequately equipped to establish the optimal time point for the administration of these antibodies prior to, during, or after the surgery, for example within a window from 3 days before through 3 days after the procedure.

Equally, the required dose, formulation, and route of application, can be determined using nothing but routine techniques.

Similarly, the passive immunisation with an isolated antibody for use according to the invention, is advantageously applied to a human or animal target shortly before, during, or immediately following a visit to a foreign country where the risk of S. aureus infection from hospital acquired, or community acquired infection is considerable.

Therefore in a preferred embodiment, the isolated antibody for use according to the invention, is applied to a human or animal subject prior to, during, or after a visit to a foreign country where the risk of S. aureus infection is considerable.

Such application is especially advantageous for those humans or animals that are more at risk of infection than others, for example for being immune-compromised in any way.

In a preferred embodiment, the isolated antibody for use according to the invention is a monoclonal antibody, a humanised antibody, a chimeric antibody, or a synthetic antibody.

Still a further advantageous variation on a use for the invention as described above, is one wherein the SSL3 protein, the homolog, or the immunogenic fragment, all for use according to the invention, are provided by a nucleic acid that can encode the SSL3 protein, the homolog, or the immunogenic fragment, all for use according to the invention. Typically the nucleic acid is a DNA molecule, as these generally are more stable than RNA molecules. However methods to produce very stable RNA's are commonly being applied.

When using DNA, such an approach is DNA vaccination', wherein a DNA molecule comprising a nucleotide sequence encoding the desired protein is administered to a human or animal target. The DNA is taken up into host cells, often dendritic cells, and transported to the nucleus where it is expressed. The protein produced is presented on the surface of the host cell to the target's immune system. Because such presentation is in the context of MHC1, this way of vaccination can generate an immune response of a different signature than that from protein based immunisation.

The DNA can be administered in a variety of ways, and can be in different forms: either as naked DNA or attached to, or encapsulated in, a carrier, for example gold-particles, when using the well known Genegun™.

Direct vaccination with DNA encoding a vaccine antigen has been successful for many different proteins, as reviewed in e.g. Donnelly et al. (1993, The Immunologist, vol. 2, p. 20). This approach has also been applied for S. aureus vaccination and was tested in mice (Arciola et al., 2009, Int. J. of Artif. Organs, vol. 32, p. 635), and bovines (Carter & Kerr, 2003, J. of Diary Scie., vol. 4, p. 1177; Shkreta et al., 2004, Vaccine, vol. 1, p. 114).

Therefore in a further aspect the invention relates to an isolated nucleic acid capable of encoding an SSL3 protein, a homolog of said SSL3 protein, or an immunogenic fragment of either protein, for use in a vaccine against S. aureus.

The concept of a nucleic acid being “capable of encoding” a protein is well known in the art, and relates to the central dogma of molecular biology on gene-expression and protein production: a nucleotide sequence on DNA is transcribed into RNA, and the RNA is translated into a protein. Typically a nucleic acid capable of encoding a protein is called an ‘open reading frame’ (ORF), indicating that no undesired stop-codons are present that would prematurely terminate the translation into protein. The nucleic acid may be a gene (i.e. an ORF encoding a complete protein), or be a gene-fragment. It may be of natural or synthetic origin.

To allow its expression, a nucleotide sequence needs to be provided with the proper regulatory signals to initiate transcription and translation, for instance being operatively linked to a promoter and a stop codon when the nucleic acid is a DNA; or to a polyA tail when the nucleic acid is an mRNA.

Routinely a nucleic acid such as for use according to the invention, is manipulated in the context of a vector, such as a DNA plasmid, enabling the amplification in e.g. bacterial cultures, and the manipulation in a variety of molecular biological techniques. A wide variety of suitable plasmid vectors is available commercially.

This way modifications can be made to the inserted nucleic acid e.g. insertions, deletions, or mutations, using common techniques of restriction enzyme digestion or by polymerase chain reaction (PCR). The resulting molecule is than a recombinant DNA molecule for use according to the invention.

For example, for the purpose of improvement of expression level, or to make the expressed protein more immunogenic, the sequence may be mutated or additional nucleotide sequences may be added. A well known modification is for instance codon optimisation; this involves the adaptation of a nucleotide sequence encoding a protein to encode the same amino acids as the original coding sequence, be it with other nucleotides; i.e. the mutations made are essentially silent. This can improve the level at which the coding sequence is expressed in a biological context that differs from the origin of the expressed gene. In practice this will mean that while most amino acids will remain the same, the encoding nucleotide sequence may differ considerably (up to 25% identity difference) from the original sequence. An alternative modification is by peptidomimetics, which can make a protein a more stable and effective vaccine (Croft & Purcell, 2011, Expert Rev. Vacc., vol. 10, p. 211).

The addition of (coding) sequences may result in the final nucleic acid being larger than the sequences required for encoding an SSL3 protein, a homolog, or an immunogenic fragment, all for use according to the invention. Upon expression such additional elements become an integral part of the expressed protein, which is then a ‘fusion protein’, for use according to the invention.

A preferred fused protein for the invention is one as described in WO2004/007525: by attaching a hydrophobic peptide to a core protein, the fusion protein more efficiently interacts with free saponin as an adjuvant. Examples of such hydrophobic peptides for fusion are described, for example a C-terminal section of decay accelerating factor (CD55).

The relevant molecular biological techniques are explained in great detail in standard text-books like Sambrook & Russell: “Molecular cloning: a laboratory manual” (2001, Cold Spring Harbour Laboratory Press; ISBN: 0879695773); Ausubel et al., in: Current Protocols in Molecular Biology (J. Wiley and Sons Inc, NY, 2003, ISBN: 047150338X); C. Dieffenbach & G. Dveksler: “PCR primers: a laboratory manual” (CSHL Press, ISBN 0879696540); and “PCR protocols”, by: J. Bartlett and D. Stirling (Humana press, ISBN: 0896036421).

An efficient way to administer an isolated nucleic acid for use according to the invention to a human or animal target, is by its incorporation in a recombinant carrier micro-organism (RCM). When alive this can safely and effectively enter, replicate, and survive the in target human or animal. But, when alive or inactivated, the RCM acts as delivery vehicle for the SSL3 protein, the homolog, or the immunogenic fragment for use according to the invention, to the host's immune system, and in that way vaccinate the host.

Therefore, in a further aspect, the invention relates to a recombinant carrier micro-organism (LRCM) for use in a vaccine against S. aureus, said RCM comprising an isolated nucleic acid for use according to the invention.

The RCM may be alive or inactivated.

When the RCM is alive, it can replicate in the vaccinated host. This route of delivery of the nucleic acid for use according to the invention may be more effective than by DNA vaccination, because expression from a replicating micro-organism is closer to the natural way of expression of the S. aureus SSL3 and SSL4 proteins. A further advantage of a live RCM is their self-propagation, so that only low amounts of the recombinant carrier are necessary for an immunisation.

Therefore, in a preferred embodiment, the RCM for use according to the invention is a live recombinant carrier micro-organism (LRCM) for use in a vaccine against S. aureus, said LRCM comprising an isolated nucleic acid for use according to the invention.

LRCMs suitable for the use according to the invention are micro-organisms that can replicate in a human or animal host, which are not (too) pathogenic to the host, and for which molecular biological tools are available for their recombination and manipulation. The LRCM can for example be a virus, a bacterium, or a parasite. Many examples of such uses are known. In humans: adenovirus, and in lifestock animals a wide variety of LRCMs have been described and are being applied: bovines: Toxoplasma theileri, bovine herpes virus (IBR); Swine: pseudorabiesvirus; dog: canine parvovirus; chicken: Salmonella, herpesvirus of turkeys, etc.

For the construction of an LRCM the well known technique of in vitro homologous recombination can be used to stably introduce a nucleic acid for use according to the invention into the genome of an LRCM. Alternatively the nucleic acid can be introduced into an LRCM for transient or episomal expression.

As described above, the SSL3 protein, the homolog, the immunogenic fragment, the isolated antibody, the isolated nucleic acid, and the LRCM, all are advantageously employed for use according to the invention, in a vaccine against S. aureus.

Therefore, in a further aspect, the invention relates to a vaccine against S. aureus comprising the SSL3 protein, the homolog of said SSL3 protein, the immunogenic fragment of either of these proteins, the isolated antibody, the isolated nucleic acid, or the LRCM, all for use in a vaccine against S. aureus, or a combination of any one thereof, and a pharmaceutically acceptable carrier.

An even more effective version of the vaccine can be devised by using more than one of the elements of the vaccine according to the invention, in combination. For example: the SSL3 protein and the homolog (e.g. an SSL4 protein) combined in one formulation. Alternatively a priming vaccination with the nucleic acid, or with the LRCM, followed later in time by a booster vaccination with the SSL3 protein and/or the homolog, etc. Such improvements and modifications are well within the routine capabilities of the skilled person.

The term “vaccine” implies the presence of an immunologically effective amount of one compound and the presence of a pharmaceutically acceptable carrier.

What constitutes an immunologically effective amount for the vaccine according to the invention is dependent on the desired effect and on the specific characteristics of the vaccine that is being used. Determination of the effective amount is well within the skills of the routine practitioner, for instance by monitoring the immunological response following vaccination, or after a challenge infection, e.g. by monitoring the targets' clinical signs of disease, serological parameters, or by re-isolation of the pathogen, and comparing these to responses seen in unvaccinated targets.

A ‘vaccine’ is well known to be a composition comprising an immunologically active compound, in a pharmaceutically acceptable carrier. The ‘immunologically active compound’, or ‘antigen’ is a molecule that is recognised by the immune system of the target and induces an immunological response. The response may originate from the innate or the acquired immune system, and may be of the cellular and/or the humoral type.

A ‘vaccine’ induces an immune response that aids in preventing, ameliorating, reducing sensitivity for, or treatment of a disease or disorder resulting from infection with a micro-organism. The protection is achieved as a result of administering at least one antigen derived from that micro-organism. This will cause the target animal to show a reduction in the number, or the intensity, of clinical signs caused by the micro-organism. This may be the result of a reduced invasion, colonization, or infection rate by the micro-organism, leading to a reduction in the number or the severity of lesions and effects that are caused by the micro-organism or by the target's response thereto.

Apart from the clear benefits a vaccine according to the invention will provide for the vaccinee itself, there are even other and further advantages to be had: for the farmer the reduction of costs resulting from sick and underproductive animals; to a human- or veterinary clinic, a reduction in number of (MRSA) S. aureus infected patients reduces the need for quarantine measures, and repeated rigorous decontamination of equipment and facilities; and for the population in general, a reduction in S. aureus carriers reduces their potential contamination and spread to others.

A “pharmaceutically acceptable carrier” is intended to aid in the effective administration of a compound, without causing (severe) adverse effects to the health of the target human or animal to which it is administered. A pharmaceutically acceptable carrier can for instance be sterile water or a sterile physiological salt solution. In a more complex form the carrier can e.g. be a buffer, which can comprise further additives, such as stabilisers or conservatives. Details and examples are for instance described in well-known handbooks e.g.: such as: “Remington: the science and practice of pharmacy” (2000, Lippincot, USA, ISBN: 683306472); “Veterinary vaccinology” (P. Pastoret et al. ed., 1997, Elsevier, Amsterdam, ISBN 0444819681); and the Merck Index, Merck & Co., Rahway, N.J., USA.

In a preferred embodiment, the compounds used for the production of the vaccine according to the invention are serum free (without animal serum); protein free (without animal protein, but may contain other animal derived components), animal compound free (ACF; not containing any component derived from an animal); or even ‘chemically defined’, in that order of preference.

In a further preferred embodiment the vaccine according to the invention additionally comprises a stabiliser.

Often, a vaccine is mixed with stabilizers, e.g. to protect degradation-prone components from being degraded, to enhance the shelf-life of the vaccine, and/or to improve freeze-drying efficiency. Generally these are large molecules of high molecular weight, such as lipids, carbohydrates, or proteins; for instance milk-powder, gelatine, serum albumin, sorbitol, trehalose, spermidine, Dextrane or polyvinyl pyrrolidone, and buffers, such as alkali metal phosphates.

Preferably the stabiliser is free of compounds of animal origin, or even: chemically defined, as disclosed in WO 2006/094,974.

Also preservatives may be added, such as thimerosal, merthiolate, phenolic compounds, and/or gentamicin.

For reasons of e.g. stability or economy, the antigen according to the invention may be freeze-dried. In general this will enable prolonged storage at temperatures above zero ° C., e.g. at 4° C.

Procedures for freeze-drying are known to persons skilled in the art, and equipment for freeze-drying up to industrial scale is available commercially.

Therefore, in a preferred embodiment, the vaccine according to the invention is in a freeze-dried form.

To reconstitute a freeze-dried vaccine composition, it is suspended in a physiologically acceptable diluent. This is commonly done immediately before use, to ascertain the best quality of the vaccine. The diluent can e.g. be sterile water, or a physiological salt solution. The diluent to be used for reconstituting the vaccine can itself contain additional compounds, such as an adjuvant. In a more complex form it may be suspended in an emulsion as outlined in EP 382.271

In a variant embodiment of the freeze dried vaccine according to invention, the diluent or adjuvant for the vaccine is supplied separately from the container comprising the freeze dried cake comprising the rest of the vaccine. In this case, the freeze dried vaccine cake and the adjuvated diluent composition form a kit of parts for the invention.

Therefore, in a preferred embodiment of the freeze dried vaccine according to the invention, the freeze dried vaccine is comprised in a kit of parts with at least two types of containers, one container comprising the freeze dried vaccine, and one container comprising an aqueous or oily diluent comprising a buffer and optionally an appropriate adjuvant.

The kit may be comprised in a box with instructions for use, which may for example be written on the box containing the constituents of the kit; may be present on a leaflet in that box; or may be viewable on, or downloadable from, an internet website from the manufacturer, or the distributor of the kit, etc.

For the invention, the kit may also be an offer of the mentioned parts (relating to commercial sale), for example on an internet website, for combined use in vaccination for the invention.

Preferably the freeze-dried vaccine is in the form as disclosed in EP 799.613.

The vaccine according to the invention may additionally comprise a so-called “vehicle”. A vehicle is a compound to which the proteins, protein fragments, nucleic acids or parts thereof, cDNA's, recombinant molecules, live recombinant carriers, and/or host cells according to the invention adhere, without being covalently bound to it. Such vehicles are i.a. bio-microcapsules, micro-alginates, liposomes, macrosols, aluminium-hydroxide, -phosphate, -sulphate or -oxide, silica, Kaolin®, and Bentonite®, all known in the art. An example is a vehicle in which the antigen is partially embedded in an immune-stimulating complex, the so-called ISCOM® (EP 109.942, EP 180.564, EP 242.380). In addition, the vaccine according to the invention may comprise one or more suitable surface-active compounds or emulsifiers, e.g. Span® or Tween®.

The age, weight, sex, immunological status, and other parameters of the humans or animals targeted to receive the vaccine according to the invention, are not critical. Nevertheless, it is evidently favourable to vaccinate healthy targets, and to vaccinate as early as possible to prevent any field infection, as long as the target is susceptible to the vaccination.

Target subjects for the vaccine according to the invention may be healthy or diseased, and may be seropositive or -negative for S. aureus antigen or antibodies.

The vaccine according to the invention can equally be used as prophylactic and as therapeutic treatment, and interferes both with the establishment and/or with the progression of an S. aureus infection or its clinical signs of disease.

The vaccine according to the invention can effectively serve as a priming vaccination, which can later be followed and amplified by a booster vaccination.

The scheme of the application of the vaccine according to the invention to the target can be in single or multiple doses, which may be given at the same time or sequentially, in a manner compatible with the dosage and formulation, and in such an amount as will be immunologically effective.

The protocol for the administration of the vaccine according to the invention ideally is integrated into existing vaccination schedules of other vaccines.

The vaccines of the invention are advantageously applied in a single yearly dose.

The vaccination of a bovine to prevent (the consequences of) bovine mastitis, by a vaccine according to the invention, is preferably performed in and around the period of pregnancy, so as to have the mother optimally protected in the first weeks of lactation, when the risk of S. aureus infection is greatest. Vaccination can therefore effectively be applied mid-term of the pregnancy with a booster vaccination shortly before the planned partus, e.g at 9 and at 3 weeks before partus.

A vaccine according to the invention may take any form that is suitable for administration to humans or animals, and that matches the desired route of application and the desired effect.

The vaccine according to the invention can in principle be in any suitable form, e.g.: a liquid, a gel, an ointment, a powder, a tablet, or a capsule, depending on the desired method of application to the target. Preferably the vaccine according to the invention is formulated in a form suitable for injection, thus an injectable liquid such as a suspension, solution, dispersion, or emulsion. Commonly such vaccines are prepared sterile.

Vaccines according to the invention can be administered in amounts containing between 0.1 and 1000 μg of protein per dose; or to achieve a desired target concentration of antibody in the subject's serum, such as 0.1-100 μg/ml; or between 1 and 1000 microgram of nucleic acid per dose; or between 1 and 1×10̂9 live units of LRCM per dose.

Vaccines according to the invention, can be administered in a volume that is consistent with the target, for instance, one vaccine dose can be between 0.1 and 5 ml. Preferably one dose is between 0.5 and 2 ml.

The vaccine according to the invention can be administered to the target according to methods known in the art. For instance by parenteral applications such as through all routes of injection into or through the skin: e.g. intramuscular, intravenous, intraperitoneal, intradermal, submucosal, or subcutaneous. Alternative routes of application that are feasible are by topical application as a drop, spray, gel or ointment to the mucosal epithelium of the eye, nose, mouth, anus, or vagina, or onto the epidermis of the outer skin at any part of the body; by spray as aerosol, or powder. Alternatively, application can be via the alimentary route, by combining with the food, feed or drinking water e.g. as a powder, a liquid, or tablet, or by administration directly into the mouth as a liquid, a gel, a tablet, or a capsule, or to the anus as a suppository.

The preferred application route is by intraperitoneal application, e.g. by intramuscular, intradermal, or subcutaneous injection.

It goes without saying that the optimal route of application will depend on the specific vaccine formulation that is used, and on particular characteristics of the target human or animal.

It is well within reach of a skilled person to further optimise the vaccine of the invention. Generally this involves the fine-tuning of the efficacy of the vaccine, so that it provides sufficient immune-protection. This can be done by adapting the vaccine dose, or by using the vaccine in another form or formulation, or by adapting the other constituents of the vaccine (e.g. the stabiliser or the adjuvant), or by application via a different route.

The vaccine may additionally comprise other compounds, such as an adjuvant, an additional antigen, a cytokine, etc. Alternatively, the vaccine according to the invention can advantageously be combined with a pharmaceutical component such as an antibiotic, a hormone, or an anti-inflammatory drug.

In a preferred embodiment, the vaccine according to the invention is characterised in that it comprises an adjuvant.

An “adjuvant” is a well known vaccine ingredient, which in general is a substance that stimulates the immune response of the target in a non-specific manner. Many different adjuvants are known in the art. Examples of adjuvants are Freund's Complete and -Incomplete adjuvant, vitamin E, non-ionic block polymers and polyamines such as dextransulphate, carbopol and pyran.

Furthermore, peptides such as muramyldipeptide, dimethylglycine, tuftsin, are often used as adjuvant, and mineral oil e.g. Bayol® or Markol®, vegetable oils or emulsions thereof and DiluvacForte® can advantageously be used.

Preferred adjuvant for the vaccine according to the invention is Saponin, more preferably Quil A®. Saponin adjuvant is preferably comprised in the vaccine according to the invention, at a level between 10 and 10.000 μg/ml, more preferably between 100 and 500 μg/ml. Saponin and vaccine components may be combined in an ISCOM® (EP 109.942, EP 180.564, EP 242.380).

For human vaccination preferred adjuvants are: aluminum hydroxide; aluminum phosphate, aluminum hydroxyphosphate sulfate or other salts of aluminum; calcium phosphate; DNA CpG motifs; monophosphoryl lipid A; cholera toxin; E. coli heat-labile toxin; pertussis toxin; muramyl dipeptide; Freund's incomplete adjuvant; MF59; SAF; immunostimulatory complexes; liposomes; biodegradable microspheres; saponins; nonionic block copolymers; muramyl peptide analogues; polyphosphazene; synthetic polynucleotides; lymphokines such as IFN-γ; IL-2; IL-12; and ISCOMS.

The vaccine according to the invention may be formulated with the adjuvant into different types of emulsions: water-in-oil, oil-in-water, water-in-oil-in-water, etc. The emulsion can be prepared at the manufacturer, and shipped ready for use, or can be mixed by a practitioner shortly before use, so-called: ‘emulsion on the spot’.

It goes without saying that other ways of adjuvating, adding vehicle compounds or diluents, emulsifying or stabilizing a vaccine are also within the scope of the invention. Such additions are for instance described in the well-known handbooks (supra).

The vaccine according to the invention has proven to be highly effective against S. aureus in bovine mastitis. In a vaccination-challenge assay, the vaccine could reduce symptoms of disease, and reduced the number of bacteria encountered in udder and milk from a severe challenge infection, after 2 vaccinations.

Therefore in a preferred embodiment, the vaccine according to the invention is applied in the prevention of bovine mastitis.

The vaccine according to the invention can advantageously be combined with another antigen.

Therefore, in a more preferred embodiment the vaccine according to the invention is characterised in that it comprises an additional immunoactive component.

The “additional immunoactive component” may be an antigen, an immune enhancing substance, and/or a vaccine; either of these may comprise an adjuvant.

The additional immunoactive component when in the form of an antigen may consist of any antigenic component of human or veterinary importance. It may for instance comprise a biological or synthetic molecule such as a protein, a carbohydrate, a lipopolysacharide, a nucleic acid encoding a proteinaceous antigen. Also a host cell comprising such a nucleic acid, or a live recombinant carrier micro-organism containing such a nucleic acid, may be a way to deliver the nucleic acid or the additional immunoactive component. Alternatively it may comprise a fractionated or killed micro-organism such as a parasite, bacterium or virus.

The additional immunoactive component(s) may be in the form of an immune enhancing substance e.g. a chemokine, or an immunostimulatory nucleic acid, e.g. a CpG motif. Alternatively, the vaccine according to the invention, may itself be added to a vaccine.

In a preferred embodiment, the vaccine according to the invention is characterised in that the additional immunoactive component or nucleotide sequence encoding said additional immunoactive component is obtained from a micro-organism infective to the human or animal target that is to be vaccinated.

The advantage of such a combination vaccine is that it not only induces an immune response against S. aureus but also against an other relevant pathogen, while only a single handling of the human or animal for the vaccination is required, thereby preventing needless stress to the target resulting from repeated handling, as well as saving time- and labour costs.

In a preferred embodiment, the additional immunoactive component for the vaccine according to the invention is an antigen from the pathogenic bacteria: H. influenzae, M. catarrhalis, N. gonorrhoeae, E. coli, and/or S. pneumoniae.

In an alternative preferred embodiment, the additional immunoactive component is a whole or a part of the S. aureus protein IsdB (Iron regulated surface determinant, also known as ORF0657n).

The preparation of a vaccine according to the invention is carried out by means well known to the skilled person.

Such vaccine manufacture will in general comprise the steps of admixing and formulation of the components of the invention with pharmaceutically acceptable excipients, followed by apportionment into appropriate sized containers. The various stages of the manufacturing process will need to be monitored by adequate tests, for instance by immunological tests for the quality and quantity of the antigens; by micro-biological tests for sterility and absence of extraneous agents; and ultimately by animal experiments for vaccine efficacy and safety. After these extensive tests for quality, quantity and sterility were all found to be compliant with the prevailing regulations, the vaccine products are released for sale.

Therefore in a further aspect the invention relates to a method for the preparation of the vaccine according to the invention, comprising the admixing of the SSL3 protein, or the homolog of said SSL3 protein, or the immunogenic fragment of either of these proteins, or the isolated antibody, or the isolated nucleic acid, or the LRCM, all for use in a vaccine against S. aureus, or a combination of any one thereof, and a pharmaceutically acceptable carrier.

The protein components of the vaccine according to the invention, the SSL3 protein, the homolog, the immunogenic fragment, and the isolated antibody, all for use according to the invention, can be obtained for use in the invention in variety of ways: e.g. by isolation from an in vitro culture of S. aureus, or from an animal infected with S. aureus. However most conveniently the proteins are produced through the use of a recombinant expression system, by the expression of a nucleic acid sequence that encodes the SSL3 protein, the homolog, or the immunogenic fragment, all for use according to the invention.

Recombinant expression systems for this purpose commonly employ a host cell, being cultured in vitro. Well known in the art are host cells from bacterial, yeast, fungal, insect, or vertebrate cell expression systems.

Therefore, in an embodiment, the invention relates to a host cell comprising a nucleic acid for use according to the invention.

The host cell for use according to the invention may be a cell of bacterial origin, e.g. from E. coli, Bacillus subtilis, Lactobacillus sp. or Caulobacter crescentus, possibly in combination with the use of bacteria-derived plasmids or bacteriophages for expressing a protein component for the vaccine according to the invention. The host cell may also be of eukaryotic origin, e.g. yeast-cells in combination with yeast-specific vector molecules (WO 2010/099186); or higher eukaryotic cells, like insect cells (Luckow et al., 1988, Bio-technology, vol. 6, p. 47) in combination with vectors or recombinant baculoviruses; or plant cells in combination with e.g. Ti-plasmid based vectors or plant viral vectors (Barton et al., 1983, Cell, vol. 32, p. 1033); or mammalian cells like Hela cells, Chinese Hamster Ovary cells, or Madin-Darby canine kidney-cells, also with appropriate vectors or recombinant viruses.

Next to these expression systems, plant cell, or parasite-based expression systems are attractive expression systems. Parasite expression systems are e.g. described in the French Patent Application, number FR 2,714,074. Plant cell expression systems for polypeptides for biological application are e.g. discussed by Fischer et al. (1999, Eur. J. of Biochem., vol. 262, p. 810), and Larrick et al. (2001, Biomol. Engin., vol. 18, p. 87). Also genetically modified animals may be generated which can express such proteins, preferably mammalians expressing the proteins in their milk, from which they can be isolated, or which may be used directly. This is well known for rabbits, and goats.

Expression may also be performed in so-called cell-free expression systems. Such systems comprise all essential factors for expression of an appropriate recombinant nucleic acid, operably linked to a promoter that will function in that particular system. Examples are an E. coli lysate system (Roche, Basel, Switzerland), or a rabbit reticulocyte lysate system (Promega corp., Madison, USA).

As is well known in the art, a consequence of the choice for a specific expression system is the level of post-translational processing that is provided to the expressed protein; e.g. a prokaryotic expression system will not attach any glycosylation signals to the polypeptide produced, whereas insect, yeast or mammalian systems do attach N- and/or O-linked glycosylation, of increasing complexity. Also, levels of lipidation, and amidation may vary; as well as type of protein processing, depending on the proteases present. The skilled person can readily make the proper choice based on selection of the system giving the best balance of protein amount and immunological effectiveness.

The isolated nucleic acid component for the preparation of the vaccine according to the invention, can be isolated from cultures of S. aureus, however, more conveniently this is obtainable by production in and isolation from a recombinant DNA production system such as based on suitable E. coli laboratory strains, cultured at industrial scale.

Materials and methods for such procedures are well known and commercially available.

Likewise, the LRCM component for the preparation of the vaccine according to the invention, can convenient be amplified and produced at industrial scale in a variety of culturing system, suitable for the particular LRCM.

In a further aspect, the invention relates to the use of an SSL3 protein, or a homolog of said SSL3 protein, or an immunogenic fragment of either protein, for the manufacture of a vaccine against S. aureus.

In a further aspect, the invention relates to the use of an isolated antibody that can bind specifically to an SSL3 protein, or to a homolog of said SSL3 protein, or to an immunogenic fragment of either protein, for the manufacture of a vaccine against S. aureus.

In a further aspect, the invention relates to the use of an isolated nucleic acid capable of encoding an SSL3 protein, or a homolog of said SSL3 protein, or an immunogenic fragment of either protein, for the manufacture of a vaccine against S. aureus.

In a further aspect, the invention relates to the use of an LRCM comprising an isolated nucleic acid for use according to the invention, for the manufacture of a vaccine against S. aureus.

In a further aspect, the invention relates to a method of vaccination of a human or animal subject, comprising the inoculation of said subject with a vaccine according to the invention.

Methods of vaccination for the invention in principle relate to any feasible method of vaccination; many of those have been described above. Preferred method of vaccination is by intra-peritoneal application.

The invention will now be further described with reference to the following, non-limiting, examples.

### EXAMPLES

### 1. Characterisation of SSL3 and SSL4 from S. Aureus as Inhibitors of the Activity of TLR2

### 1.1. Materials and Methods

1.1.1 Antibodies

FITC-conjugated mAbs directed against CD9, CD11a, CD31, CD46, CD62L, CD66, and phycoerythrin (PE)-conjugated mAbs directed against CD35, CD44, CD47, CD49b, CD54, CD58, CD87, CD114, CDw119, CD162, and CD321, allophycocyanin (APC)-conjugated mAbs directed against CD11b, CD11c, CD13, CD14, CD29, CD45, CD50, CD55, and Alexa-647-conjugated mAb directed against CD16 were purchased from BD Bioscience. FITC-labelled mAbs against CD120a, and CD120b, and an APC-conjugated mAb against Siglec-9 were from R&D Systems. Anti-CD43-FITC was from Santa Cruz Biotechnology. Anti-LTB4R-FITC, anti-CD32-PE, and anti-CD89-PE were from AbD Serotec. Anti-CD88-PE was from Biolegend. Anti-CD282-PE was from Ebioscience. Anti-CD63-PE was purchased from Immunotech. Fluorescent formylated peptide (fluorescein conjugated of the hexapeptide N-formyl-Nle-Leu-Phe-Nle-Tyr-Lys) to detect formyl peptide receptor 1 and anti-CD10-APC were purchased from Invitrogen.

1.1.2 Cloning, Expression and Purification of SSL3 and SSL4

For expression of recombinant SSL3, the SSL3 gene of S. aureus strain NCTC 8325 (SAOUHSC_{—}00386), except for the signal sequence, was cloned into the pRSETB vector (Invitrogen) as described (Bestebroer et al., 2007, Blood vol. 109, p. 2936). After verification of the correct sequence, the pRSETB/SSL3 expression vector was transformed in Rosetta-Gami(DE3)pLysS E. coli (Novagen). Expression of histidine (His)-tagged SSL3 was induced with 1 mM isopropyl-β-D-thiogalactopyranoside (IPTG; Roche Diagnostics) for 4 h at 37° C. in LB containing 20 mM glucose. His-tagged SSL3 was isolated under denaturing conditions on a HiTrap™ chelating column, according to the manufacturer's description. Elution was performed in 50 mM EDTA under denaturing conditions. Renaturation of His-SSL3 was performed by dialysis, after which the His-tag was removed by enterokinase cleavage according to the manufacturer's instructions (Invitrogen).

Finally, the purity of SSL3 was checked by SDS-PAGE and protein was stored in PBS at −20° C. Cloning and expression of SSL 1, 2, 4, and 5 to 11 from S. aureus strains NCTC 8325 and SSL4 from strain MRSA252 was performed as described for SSL3 with minor modifications. The N-terminal histidine tag of the pRSETB vector, contains besides the histidine tag and enterokinase cleavage site also an Xpress epitope, which was replaced by a 6 residue histidine tag just downstream the enterokinase cleavage site. After enterokinase cleavage, an additional Glycine residue remains at the N-terminus of the SSL4 proteins.

1.1.3 Cells

Human neutrophils and peripheral mononuclear cells (PBMCs) were isolated as described (Bestebroer et al., 2007, supra). Human embryonal kidney cells expressing TLR2 (HEK-TLR2) and TLR2 in combination with TLR1 (HEK-TLR1/2) and TLR6 (HEK-TLR2/6) were obtained from Invivogen. HEK-TLR cell lines were maintained in DMEM, containing 10 μg/ml gentamicin, 10 μg/ml blasticidin and 10% FCS. Mouse macrophage cell line RAW264.7 was cultured in DMEM, containing 10 μg/ml gentamicin and 10% FCS.

1.1.4 SSL3 Binding to Cells

To determine binding of SSL3 to different leukocyte populations, SSL3 was labeled with fluorescein isothiocyanate (FITC). Therefore, 1 mg/ml SSL3 was incubated with 100 μg/ml FITC in 0.1 M sodium carbonate buffer (pH 9.6) for 1 hour at 37° C. A HiTrap desalting column (GE healthcare) was used to separate FITC-labeled SSL3 from unbound FITC. For binding of SSL3-FITC to leukocytes, human neutrophils (5×10⁶ cells/ml) and PBMCs (1×10⁷ cells/ml) were incubated on ice for 30 min with increasing concentrations of SSL3-FITC in RPMI (Gibco), containing 0.05% human serum albumin (Sanquin). After washing, fluorescence was measured on a flow cytometer (FACSCalibur; Becton Dickinson).

1.1.5 Competition for TLR2 Binding Between SSL3 and Antibody T2.5

To determine a putative receptor for SSL3, a mixture of neutrophils (5×10⁶ cells/ml) and PBMCs (1×10⁷ cells/ml) were incubated with either SSL3 (10 μg/ml) or RPMI/HSA and incubated 30 min on ice. Subsequently, 39 different FITC-, PE-, or APC-conjugated monoclonal antibodies (mAbs) directed against various cell-surface receptors were added to the cell mixture and incubated for 45 min on ice. After washing, fluorescence was measured using flow cytometry. Neutrophils, monocytes and lymphocytes were selected by gating. In another experiment, leukocytes were incubated with increasing concentrations of SSL3 for 30 min at 4° C. Subsequently, the cells were incubated with anti-TLR2 antibody T2.5 (anti-CD282-PE; 1:100 dilution) using the same conditions as in the screening assays.

1.1.6 TLR2 Ligand-Induced IL-8 Production

To test the effect of SSL3 on TLR2 ligand-induced IL-8 production, HEK-TLR2, HEK-TLR1/2, HEK-TLR2/6, PBMC, neutrophils, and RAW264.7 cells were used. HEK and RAW264.7 cells were seeded in 96 wells culture plates until confluency. Freshly isolated PBMC and neutrophils were added to 96 wells culture plates (2.5×10⁶ cells/well). To avoid activation of TLR4 on PBMC and neutrophils by endotoxin, SSL3 was pretreated with 20 μg/ml polymyxin B sulphate (Sigma) for 1 hour. Additionally, PBMC were preincubated with 10 μg/ml blocking anti-TLR4 mAb (clone HTA125; Bioconnect) for 30 minutes. Next, the cells were preincubated for 30 minutes at 37° C. with increasing concentrations of SSL3. Then, cells were stimulated with different, increasing concentrations of Pam2Cys, Pam3Cys (both from EMC microcollections), MALP-2 (Santa Cruz), or recombinant flagellin of P. aeruginosa (Chapter 2), as indicated in the Results section (Example 2).

After overnight incubation in a 37° C. incubator, culture supernatants were tested for presence of IL-8 using a specific ELISA following the manufacturer's instructions (Sanquin). Culture supernatants of RAW264.7 cells were tested for the presence of mouse TNFα using a specific ELISA kit (R&D systems). IL-8 production experiments with PBMC and neutrophils were performed in RPMI/10% FCS. Experiments with HEK and RAW264.7 cells were performed in DMEM/10% FCS. Cytotoxic effect of SSL3 on cells was tested using the lactate dehydrogenase (LDH) cellular cytotoxicity detection kit following the manufacturer's description (Roche Diagnostics). In some experiments, next to SSL3 and SSL4, the other SSLs of SaPI2 were tested for IL-8 production by MALP-2-activated HEK-TLR2/6 cells, as described above.

1.1.7 Cloning and Expression of Human and Mouse TLR2

The recombinant extracellular domain of human TLR2 (hTLR2) was cloned in HEK293 cells (U-Protein Express, The Netherlands). The recombinant extracellular domain of mouse TLR2 (mTLR2) was cloned and expressed by a different department (Crystal and Structural Chemistry, University Utrecht, The Netherlands) in HEK293 cells. Both hTLR2 and mTLR2 contain a N-terminal 6 residues histidine tag, a 3× streptavidin tag and a TEV cleavage site.

1.1.8 ELISA

To test binding of SSL3 to the recombinant extracellular domains of human and mouse TLR2, the TLR2 proteins were coated to an ELISA plate (Nunc maxisorp) at 10 μg/ml. Wells were blocked with 4% skimmed milk in PBS/0.05% Tween. His-tagged SSL3 was allowed to bind to the coated TLR2 proteins for 1 hour at 37° C. Bound His-SSL3 was detected with anti-Xpress™ mAb (Invitrogen) and subsequent binding of peroxidase-labeled goat anti-mouse IgG and visualized as described (Haas et al, 2004, J. of Immunol., vol. 173, p. 5704).

### 1.2. Results

1.2.1 SSL3 binds to TLR2 on neutrophils and on monocytes.

To investigate its role in immune evasion, SSL3 of S. aureus strain NCTC 8325 was cloned in E. coli. The protein was pure according to SDS-PAGE and fluorescently-labelled to study the interaction with human leukocytes. SSL3 specifically interacted with human neutrophils (FIG. 1A) and monocytes (FIG. 1B), whereas almost no binding was observed for lymphocytes (FIG. 1C).

To verify that the molecular target for SSL3 was exclusively TLR2 on phagocytes, the binding of SSL3 to other receptors that are expressed on neutrophils and monocytes, with crucial functions in innate immunity (e.g. chemotaxis, activation, adhesion, and phagocytosis), was investigated, using a panel of monoclonal antibodies (mAb) recognizing these receptors.

It was found that SSL3 specifically inhibited binding of the function-blocking TLR2 monoclonal antibody T2.5 to neutrophils and monocytes (FIG. 2A). Inhibition of other tested cell-surface receptors was not observed.

The expression of TLR2 differed between cell-types; monocytes (FIG. 2B) expressed higher levels compared to neutrophils (FIG. 2C), whereas TLR2 was absent on lymphocytes (data not shown). SSL3 dose-dependently blocked binding of anti-TLR2 to monocytes (FIG. 2B) and neutrophils (FIG. 2C). The IC50 for monocytes was around 0.05 μg/ml SSL3 and for neutrophils around 0.02 μg/ml (FIG. 2D). This slightly lower half maximal inhibitory concentration corresponds with the lower expression of TLR2 on neutrophils. These data indicate that SSL3 efficiently, and specifically, blocks a domain of TLR2 that is important for its function.

1.2.2 SSL3 Inhibits the Activation of TLR2

To test whether SSL3, next to binding, could also inhibit TLR2 function, HEK cells expressing TLR2 (HEK-TLR2) were stimulated with the synthetic lipopeptides Pam2Cys and MALP-2, and the production of interleukin-8 (IL-8) was measured. SSL3 was found to potently inhibited TLR2 activation by both agonists in a dose-dependent manner (FIGS. 3A and 3B), confirming that SSL3 functionally inhibits TLR2. At 1 μg/ml SSL3, IL-8 production was abolished even when stimulated with 100 ng/ml Pam2Cys or MALP-2. Since TLR2 can dimerise with either TLR1 or TLR6 and thereby can discriminate between di- and tri-acylated lipoproteins and augment the cellular cytokine response, SSL3 inhibition was also tested on HEK-TLR2/6 or HEK-TLR1/2 cells activated with their specific synthetic ligands, MALP-2 (FIG. 3C) and Pam3Cys (FIG. 3D), respectively. SSL3 inhibited the IL-8 production of HEK-TLR1/2 cells, however inhibition was less potent in comparison with HEK-TLR2/6 cells.

The effect of SSL3 on TLR2 activation was also tested in primary human neutrophils and monocytes. In contrast to HEK-TLR2 cells, neutrophils and monocytes also express TLR4, which can be activated in by lipopolysaccharide that is present in recombinant proteins generated in E. coli. To prevent IL-8 production via TLR4, we pretreated SSL3 with 20 μg/ml polymyxin-B to inactivate the lipopolysaccharide contamination. Additionally, PBMCs were pretreated with 10 μg/ml blocking anti-TLR4 mAb to prevent TLR4 activation. These precautions were sufficient to block TLR4 activation in both cell types, as even the highest concentration of SSL3, without addition of MALP-2, did not induce IL-8 production (FIGS. 4A and 4B).

In addition to HEK cells overexpressing TLR2, SSL3 also efficiently inhibited TLR2 activation by MALP-2 of both neutrophils (FIG. 4A) and PBMCs (FIG. 4B), as a source for monocytes.

SSL3 was not cytotoxic for cells, as verified by a lactate dehydrogenase (LDH) cytotoxicity assay performed on PMBCs and HEK-TLR2/6 cells after overnight incubation with SSL3 (FIGS. 4C and 4D). SSL3 did not affect the IL-8 ELISA, as no difference in IL-8 standard curve was observed in the presence of 10 μg/ml SSL3 (data not shown).

The inhibition of TLR2 activation could also be obtained using a C-terminal fragment of SSL3, the fragment from amino acids 127-326 of SEQ ID NO:1, see FIG. 12.

1.2.3 SSL3 Recognizes Both Human TLR2 and Mouse TLR2

These results thus strongly suggest that SSL3 is a specific TLR2 inhibitor. It was further investigated whether SSL3 binds to the extracellular domain of TLR2 since this domain is crucial for ligand recognition and TLR2 activation. Therefore, the extracellular domains of human and mouse TLR2, expressed in HEK293 cells, were purified and tested for binding to SSL3. ELISA studies showed that SSL3 effectively and dose-dependently bound to the extracellular domains of both human and mouse TLR2 (FIG. 5A). As SSL3 efficiently bound to human as well as mouse TLR2, it was tested whether SSL3 could also inhibit the activation of TLR2 in the mouse macrophage cell line RAW264.7.

Indeed, SSL3 also functionally inhibited mouse TLR2. SSL3 potently inhibited binding of the function-blocking anti-TLR2 to RAW264.7 cells (95.6±0.95% inhibition at 0.1 μg/ml (data not shown). In addition, SSL3 completely blocked TLR2 activation by MALP-2, as measured by inhibition of TNFα production (FIG. 5B). Altogether we have shown that SSL3 is a specific and potent inhibitor of human and murine TLR2, which makes in vivo testing in mouse models feasible.

1.2.4 SSL3 Exclusively Targets TLR2

TLRs, including TLR5, induce intracellular signalling via the common adaptor protein MyD88. To exclude an effect of SSL3 on this common TLR signalling pathway downstream of TLR2, we tested whether SSL3 could inhibit TLR5 activation. Therefore, HEK-TLR5 cells were activated with flagellin a TLR5-specific ligand. Isolation of flagellin and AprA has been described (Bardoel et al., 2011, PLoS Pathog. vol. 7: e1002206. doi:10.1371/journal.ppat.1002206). Briefly, flagellin was obtained by expression of the flic gene (Swiss-prot acc. nr. P72151) of P. aeruginosa strain PAO1 in E. coli. AprA was obtained by expression of the aprA gene (Swiss-prot acc. nr. Q03023) of P. aeruginosa strain PAO1 in E. coli. Both proteins were expressed with a N-terminal 6× his-tag and purified using a His Trap™ column (GE Healthcare)

SSL3 could not inhibit flagellin-induced IL-8 production of neutrophils (FIG. 6). In contrast, AprA, which degrades flagellin and thereby prevents TLR5 activation, abolished flagellin mediated IL-8 production (FIG. 6). Polymyxin B was added to prevent TLR4 dependent IL-8 production as a result of endotoxin contamination of SSL3. Addition of only Polymyxin B to flagellin did not change the flagellin-induced activation of TLR5. As control, IL-8 production by MALP-2 was inhibited by SSL3. These results exclude that SSL3 inhibits the common MyD88-mediated intracellular signalling cascade, and confirm that SSL3 specifically acts on TLR2 itself.

1.2.5 Lack of Affinity of Other SSLs for TLR2

SSLs present in pathogenicity island SAPI2 share some sequence and structural elements. It was therefore tested whether SSL1 to 11, all from S. aureus strain NCTC 8325 could, could inhibit TLR2 activation, as observed for SSL3. However, none of the other SSLs, except for SSL4, inhibited the MALP-2 induced IL-8 production by HEK-TLR2 cells using a concentration of 10 μg/ml (FIG. 7A).

To check the TLR2 inhibiting activity of both SSL4 variants, we analyzed the effect of both proteins on HEK-TLR2/6 cells activated with MALP-2. SSL4-MRSA was about 10-fold more active then SSL4-8325, which correlates with the higher homology to SSL3 in the amino acid sequence alignment (FIG. 7B). However, SSL4-8325 (FIG. 7B) was still about 30-fold less active then SSL3-8325 (FIG. 3B). In conclusion, the TLR2 inhibiting properties of SSL3 reside within its C-terminal domain. See Table 5.

1.2.6 Further Details

Further details on the characterisation of SSL3, including its specific binding and inhibition of TLR2 are presented in Bardoel et al., 2012, J. of Mol. Med., epub 20 Jun. 2012, DOI 10.1007/s00109-012-0926-8, and its supplemental data file.

### 2. Seroresponse Against SSL3, Homolog, and Fragment, in Healthy Subjects

The presence of antibodies against SSL3 protein, against a homolog, and against a fragment, all according to the use for the invention, in healthy human volunteer sera was tested. All sera were found to be decidedly positive for all three proteins.

### 2.1. Method

The sera of 36 apparently healthy human volunteers were tested individually for the presence of specific IgG antibodies directed against: SSL3 protein (from S. aureus, strain NCTC 8325; SEQ ID NO 1); against a fragment of SSL3 protein (from S. aureus, strain NCTC 8325; SEQ ID NO 1—amino acid numbers 127-326); and against a homolog of SSL3: SSL4 protein (from S. aureus, strain NCTC 8325; SEQ ID NO: 6).

The proteins had been produced as described (see Example 1.1.2).

An ELISA was performed to test the sera on the proteins: proteins were coated overnight, at 10 μg/ml of each protein in 0.1M sodiumcarbonate buffer, pH 9.6 in separate Nunc MaxiSorp™ 96 wells plates. Next day, the plates were washed 4 times with PBS/0.05% Tween and blocked for 1 hour at 37° C. with 4% skimmed milk in PBS/0.05% Tween, and then washed 4 times with PBS/0.05%. Serum was pre-diluted from 10% to 0% (1:4 dilution each step), in PBS/1% skimmed milk/0.05% Tween, and added to the wells of the plates. These were incubated with the serum samples for 1 hour at 37° C. Then, after washing the plates 4 times with PBS/0.05% Tween, incubated with peroxidase-labeled goat-anti-human IgG for 1 h at 37° C. Finally TMB-based substrate was added, and the reaction was stopped with H₂SO₄. Binding was detected by measuring absorbance at 450 nm in a BioRad ELISA-reader.

### 2.2. Results:

Data were expressed as the frequency distribution of IgG titers measured. The titer was defined as the 10 log of the dilution that gave an absorbance of 0.400 relative Elisa units, after substraction of background value. The results are represented in FIG. 8. The mean titers detected were:
SSL3 protein: 3.24 (see FIG. 8 A)
SSL3 protein fragment (127-326): 3.18 (see FIG. 8 B), and
SSL4 protein: 3.56 (see FIG. 8 C).

### 2.3. Conclusions:

All sera tested from healthy humans, possessed circulating antibodies that reacted specifically with SSL3, a fragment thereof, and a homolog thereof (SSL4). In this set of measured samples there were no titers below the detection limit of the used ELISA. As the studied population was of mixed composition, it is considered representative for the general human population.

The antibody titers detected were rather high, indicating that these SSL proteins are quite immunogenic by themselves. Moreover, this proves that SSL3 and SSL4 proteins are produced by S. aureus in vivo in amounts high enough to mount a proper antibody immune response.

### 3. Application of SSL3 as Vaccine Against S. Aureus Induced Bovine Mastitis

### 3.1. Introduction

The objective of this study is to investigate the efficacy of different S. aureus vaccines. The first vaccine will contain SSL3, the second SSL3 and an S. aureus bacterin, of killed whole cells. The third vaccine will contain SSL3 in combination with other antigens from S. aureus, and the fourth vaccine will contain SSL3 in combination with the same additional antigens, but formulated in a different adjuvant. Also a mock vaccinated group will be included.

Efficacy of the immunizations will be tested by experimental intramammary challenge infection with S. aureus Newbould 305 (ATCC 29740).

### 3.2. Experimental Design

Calved, lactating cows, will be allotted to 5 groups, each of 8 cows. After acclimatization, group 1 will be vaccinated intramuscularly with 2 ml of vaccine 2 (±100 μg of SSL3 in Alu-oil as adjuvant). Group 2 will be vaccinated intramuscularly with 2 ml of vaccine 2 (±100 μg of SSL3 and 10̂9 killed S. aureus bacteria in Alu-oil as adjuvant). Group 3 will be vaccinated intramuscularly with 2 ml of vaccine 3 (±100 μg of each antigen in Alu-oil as adjuvant). Group 4 will be vaccinated intramuscularly with 2 ml of vaccine 4 (±100 μg of each antigen, in a different adjuvant than used for vaccine 3). Group 5, is the mock-vaccinated control group (receiving only the empty Alu-oil emulsion).

The vaccination of groups 1 to 5 will be repeated after 5 weeks with a booster vaccination. Cows of all groups 1-5 will be vaccinated intramuscularly into the neck; the first vaccination into the right side of the neck, and the second vaccination in the left side.

Two homolateral quarters per cow will be intramammarily challenged with ±2000 CFU/quarter 4 weeks after the second vaccination. Efficacy of the vaccine is evaluated by monitoring the course of the intramammary infections before and after challenge. The course of infection is determined by bacteriological examination, counts of colonies of S. aureus, and the level of somatic cell counts in fore milk. Antibody titers against the sub-units and/or whole cells in serum and/or milk will also be determined at several time points during the course of the experiment.

### 3.3. Biosafety of Challenge Material:

Staphylococcus aureus is an EC class 2 organism with a broad host range spectrum including men (zoonosis). S. aureus Newbould 305 (ATCC #29740) will be used as challenge strain. This strain was isolated on Jun. 6, 1958, from a clinical case of mastitis in a cow at Orangeville, Ontario, Calif. It was coagulase-positive and alpha-beta haemolytic. The strain was tested to be sensitive to penicillin, dimethoxphenyl penicillin, dihydrostreptomycin, tetracycline and chloramphenicol.

To prevent risk of zoonotic infection, direct contact of the skin with milk and animals after challenge is to be avoided, by using appropriate personal safety equipment and following prescribed procedures.

### 3.4. Materials and Methods

3.4.1 Vaccines

The antigen part of the vaccines will be recombinant proteins and/or killed S. aureus cells and the adjuvant will be Alu-oil, or an oily adjuvant; ±100 μg of each antigen per vaccine dose and/or 10⁹ S. aureus cells per vaccine dose. The total volume of the vaccine will be 2.0 ml and applied intramuscular. Vaccine will be stored at +2 to +8° C.

3.4.2 Preparation of the Vaccine

The SSL3 protein has been expressed as described in Example 1.1.2. The S. aureus killed cells, will be prepared from a fresh culture S. aureus Newbould 305 (ATCC 29740), grown in trypticase soy broth (TSB, BioTrading) diluted at 1.0×10̂9 CFU/ml in 0.9% NaCl solution. Cells will be killed by adding 0.25% BPL (RT, 24 hours).

After incubation cells will be pelleted and taken up in 0.9% NaCl solution with a final concentration of 10¹⁰ cells per ml.

### 3.4.3 Preparation of the Challenge Material

The challenge strain is kept freeze-dried at 5° C. Two days before inoculation, the strain will be cultured on blood agar base plates in duplo overnight at 37° C. The strain will be checked for purity. Three colonies will be subcultured overnight at 37° C. in trypticase soy broth in independable duplo's. One culture will be used for preparing the final inoculum. For this final inoculum bacteria will be washed one time (2000×g, RT, 10 min.) in 0.9% physiological saline. Based on a total cell counting (in duplo, by one person), washed bacteria will be resuspended in 0.9% physiological saline to yield approximately ±2000 CFU/ml. Before and after challenge viable cell counting of the final inoculum will be performed in duplo. Challenge material will be transported at RT.

### 3.5. Test System

### Animals:

Clinically healthy, lactating heifers will be used, in five groups of eight heifers.

### Age and Parity:

All heifers have calved for the first time before the experiment; and will be between 1.5 and 3 years old at the start of the experiment.

### Clinical Condition:

the heifers will undergo a veterinary examination before the experiment, and any observations will be reported; only clinically healthy cows will be used. During selection of the cows for use in the experiment, special attention will be paid to the absence of udder or teat lesions, and animal history of mastitis. If needed, heifers will be treated with appropriate antibiotics.

### Identification:

the heifers will be identified with a unique number using a leg collar

### Treatments and Vaccinations:

A veterinarian will be responsible to decide if the cows need treatments before acclimatization, e.g. treatments against mange, prophylactic treatment with a magnet against traumatic reticulitis. Treatments will be recorded.

### Acclimatization:

the acclimatization period will be at minimum of 7 days before start of vaccination.

### Housing:

the animals will be housed in a free stable with 2×5 herringbone milking parlour.

### Food and Water:

Food will be provided according to standard protocol; water is available ad libitum.

### Milkings:

the cows will be milked two times daily in the morning and afternoon. Milk yield will be determined with transparent recorder jars. Teat dipping will be performed after milking.

### 3.6. Grouping and Dosing

### Assignment of Animals to Treatment Groups:

the cows will be allotted to 5 groups of 8 cows based upon days in lactation, mastitis history, SCC and other parameters.

3.6.1 Treatment Schedule

### Vaccinations:

The animals in the vaccination groups (1-4) will receive two doses of vaccine with an interval of 5 weeks. The vaccines will be injected intramuscular into the neck; 1^{st} dose (2 ml) at the right side and the 2^{nd} dose (2 ml) at the left side. The vaccinations will be executed according to standard procedure, and will be recorded.

### Challenge:

Cows will be challenged ±4 weeks after the second vaccination. However, before challenge, milk of all cows should be negative for antibiotic residues. All cows will receive intramammary inoculations into two homo-lateral, pathogen free quarters per cow. Prior to inoculation the teat end will be thoroughly disinfected with 70% alcohol. Inoculations will be performed by infusion of 1.0 ml of inoculum (±2000 CFU per quarter) into the teat cistern of 2 milked-out mammary quarters per cow. Infusions will be performed after the morning milking with sterile plastic 2 ml-syringes and individual plastic infusion canulas. All quarters to be inoculated will be checked for the presence of major mastitis pathogens on at least two a.m. milkings prior to inoculation and the number of somatic cell counts present will be determined at the same time. Major mastitis pathogens are Staphylococcus aureus, Streptococcus dysgalactiae, Streptococcus agalactiae, Streptococcus uberis and coliform bacteria. Challenge will be recorded.

3.6.2 Experimental Procedures and Parameters

### General Veterinary Examination

A general veterinary examination will be performed at 1 to 7 days before first vaccination. Moreover, a general veterinary examination will be carried out in case of systemic illness. Observations will be recorded.

### Daily Observations

The heifers will be observed once daily during the first part of the experiment for general health, physical appearance, behaviour, aspect of faeces and appetite. Observations will be recorded. In case of abnormalities the responsible veterinarian will be consulted.

After challenge the animals will be observed twice daily at morning and evening milkings. In case of abnormalities observations will also be recorded.

### Milk Yield

The total daily milk yield will be determined during the entire experiment and recorded.

### Udder and Milk Score

After the challenge the udder and milk scores will be assessed per quarter once daily at the morning milking for the remaining of the entire experiment according to the following scheme

Udder Scores:
0=soft pliable udder, no abnormalities
1=slight swelling,
2=moderate swelling,
3=severe swelling,
4=other abnormalities (specify)

Milk Scores:
0=normal milk
1=milk with some flakes or clots (<10)
2=milk with many flakes or clots 10)
3=serous, watery milk
4=other abnormalities (specify)

In case the milk or udder score is ≧0, then the score will be recorded; in case the milk score is once ≧2, or the milk score is ≧1 at two consecutive milkings, bacteriological examination of foremilk will be performed.

### Bacteriological Examination and Somatic Cell Counts of Foremilk Samples

Separate foremilk samples for determination of somatic cell count (SCC), for bacteriological examination and other (cellular and complement) assays will be collected according to the time schedule. The samples for bacteriological examination (5 ml) will be collected from each quarter into plastic tubes with screw caps (Sterilon). The samples for SCC will be collected into plastic tubes with Na-azide.

Samplings will be before milking according to the following procedure:
clean the teat according standard procedures (cloth, alcohol)
discard 2 squirts of milk;
collect milk sample for bacteriological examination
collect milk sample for antibody and cellular assays;
collect milk sample for somatic cell count;
perform teat dipping with teat dip after milking.

Samplings will be recorded

### Blood Sampling:

Blood for the various assays will be collected from the jugular or cocygeal vein in serum tubes (4 tubes each time). The blood for serum will be collected once every week during the whole experiment. Samplings will be recorded.

### Storage and Transport of Samples

Samples will be stored at +2 to +8° C. (cell counts, bacteriological examination and other assays) up to transport to a microbiological laboratory for bacteriological analysis. During transport the samples are kept at ambient temperature.

### Bacteriological Examination

Bacteriological examination will be started within 4 hours after collection when samples are collected at morning milking and within 18 hours when collected at evening milking. Milk (50 μl) will be plated on blood agar and incubated at 37° C. during 16-24 hours. Bacteria will be presumptively identified by colony size, morphology, pigmentation, type of haemolysis and identified further using Gram-stain, coagulase test (Staphylococcus aureus) and biochemical tests.

### Determination of Milk Somatic Cell Counts

SCC in foremilk samples from each quarter will be determined using the Fossomatic method at the Central Milk Control Lab.

The blood samples that will be processed and used for antibody ELISA and cytokine assay will be collected into 4 serum vacutainers.

### 3.7. Evaluation of Results

Data obtained by general observations, milk yield, and milk and udder scores, will supply basic information on each individual cow. Data on the presence of S. aureus in milk and on the SCC will supply information on the efficacy of treatments. Data on the presence of antigen specific antibodies, cytokine profile and phagocytosis in the presence of serum will supply information on the quality of the vaccinations, type of immune response and feasibility of the current approach.

### 3.8. References

National Mastitis Council: Microbiological procedures for the diagnosis of bovine udder infection, 3^{rd} ed., 1990.

### 4. Results from Vaccination-Challenge Experiment of Example 3

The experiment of Example 3 was performed essentially as described, with minor modifications: one group of heifers received a combination vaccine comprising SSL3 and a bacterin, and one group received a mock vaccination of an empty adjuvant formulation. The bacterin part of the SSL3 vaccine was made up of 1×10̂10 S. aureus cells, which had been inactivated with 0.5% formalin. Challenge was done with about 1000 Cfu's per quarter.

### 4.1. Challenge Protection Results

The SSL3 comprising vaccine was found to provide a strong and effective immune protection against S. aureus challenge: main effect observed was a strong reduction of the number of S. aureus challenge bacteria that could be re-isolated out of the milk from the vaccinated group. Over a period of 6 weeks after challenge (10 time points) the average cfu's re-isolated per infected udder quarter per time point, was 175 for the vaccinated animals and 6882 for the mock vaccine group. This represents a reduction of approximately 40-fold, or: a 97% reduction.

Also the somatic cell count (SCC) was reduced in the milk of vaccinated and challenge-infected quarters, when compared to mock-vaccinated challenge-infected quarters. In the period of 1 to 6 weeks after challenge, 45% of milk samples showed a SCC lower than 100,000 in vaccinated animals (average: 456,385), while in mock vaccine treated animals only 22% of milk samples was below 100,000 (average: 597,250). This corresponds to a 24% reduction of SCC resulting from vaccination with the SSL3 vaccine.

Milk yield, milk scores, and udder scores did not show significant differences between SSL3 vaccinated and mock vaccinated groups.

### 4.2. SSL3-Specific TLR2-Binding Interference by Anti-SSL3 Antibodies

Proof was also obtained that the anti-SSL3 antibodies that were induced in the cows by the vaccination, were capable of specific binding to SSL3, and thereby preventing SSL3 from binding to TLR2.

This was tested in a competition-inhibition assay, essentially as described in Example 1.1.5 above and in Bardoel et al., 2012 (supra). In short, the experimental design was based on detecting whether SSL3-specific antibodies were present in the cow sera, by detecting their binding to a set amount of SSL3. Therefore, cow sera from before and after vaccination were compared. These sera were incubated with a fixed amount of SSL3 protein. Any anti-SSL3 antibodies (when present) would then bind to SSL3 protein which would prevent the SSL3 from binding to a TLR2 receptor that was provided by expression on the surface of recombinant HEK cells. When unbound, the SSL3 would bind the TLR2 which would prevent a fluorescently labelled antibody against TLR2 (PE-labelled antibody clone T2.5, EBioscience) to bind to the cells. The resulting fluorescence intensity of the HEK cells was then detected by flow-cytometry. SSL3 protein was produced as described in Example 1, §1.1.2 above; HEK 293T-TLR2/6 cells are described above in §1.1.7.

In this assay the fluorescence levels measured on HEK-TLR2/6 cells after wash, are reduced by the presence of SSL3, when anti-SSL3 antibodies are absent; or vice versa: when SSL3-binding antibodies are present in the cow sera, SSL3 was covered with antibody which prevented its binding to TLR2, allowing the anti-TLR2-PE antibodies to bind to the TLR2-expressing HEK cells, and the fluorescence level measured remained as high as in the control sample, without SSL3.

For each cow one serum from before vaccination was tested, and one from after vaccination and each serum was tested with and without SSL3. Consequently there were 4 samples for each cow in the experiment: pre-vac, pre-vac+SSL3; post-vac; and post-vac+SSL3.

4.2.1 Details of the Competition-Inhibition Assay:

The cow sera from pre-vaccination were taken just before the first vaccination, and the post-vaccination sera just before the challenge. The sera were heated at 56° C. for 30 min. to inactivate complement. A preparation of wild type S. aureus SSL3 protein was diluted in RPMI medium to reach a concentration of 0.3 μg/ml in the final incubation sample. Next 10 μl of RPMI medium (RPMI 1640 with 0.05% w/v human serum albumin) with or without SSL3 was pipetted into wells of a 96-well plate. Then 5 μl of inactivated cow serum dilution was added to the wells to reach 10% final concentration, from either pre-vac or post-vac serum. Plates were incubated for 30 minutes at room temperature. Next HEK293T-TLR2/6 cells were added in 30 μl, to an amount of about 100,000 cells/well. This was incubated for another 30 minutes, on ice. Plates were centrifuged for 5 min at 1200 rpm, 4° C., to stick the cells to the bottom, and washed twice. Then 50 μl of TLR-2 antibody-PE (diluted 1:100) was added to each well, and plates were incubated for 45 min. on ice, in the dark. Plates were centrifuged and washed, and the cell pellets were resuspended in 200 μl RPMI medium and measured in a flow cytometer (BD FACS Calibur®), with specific voltage setting for the required channels.

4.2.2 Results of Competition-Inhibition Assay:

The results of these competition-inhibition assays for the sera from the experiment of Examples 3 and 4 are presented in FIG. 13: panel A presents the results from the cow sera from the mock-vaccinated group, and panel B from the SSL3 vaccinated group. The columns represent the fluorescence intensity measured for the different serum samples: pre- and post-vac, and with or without SSL3. Fluorescence levels are presented as averages per group, with standard deviation bars, whereby p=0.05 and n=8 for the mock vaccinated group (panel A), and n=7 for the SSL3 vaccinated group (panel B).

FIG. 13 A displays that all controls were as expected: the column heights are essentially equal for the pre- and post-vac sera without SSL3, and both were strongly reduced when SSL3 was present. However this is different in the last column of panel B (sample post-vac+SSL3), where the fluorescence remains essentially unchanged even though SSL3 had been added: this proves that SSL3-specific antibodies were present in these cow sera, and that these sera could prevent SSL3 protein from binding to TLR2.

### 4.3. Conclusions

In conclusion, the vaccination with an SSL3 protein-containing vaccine induces in cows a strong immune response that helps the cows to effectively suppress a severe intra-mammary challenge infection with S. aureus. The efficacy of this vaccine could be ascribed to SSL3-specific antibodies which were present in SSL3 vaccinated cow sera, but not in mock-vaccinated cow sera. This was demonstrated by a competition-inhibition assay, which centred on the capability of these SSL3-specific antibodies to prevent SSL3 protein, by their specific binding, to interact with a TLR2 receptor. This interferes with S. aureus' capability to evade the host's (native) immune response and establish its infection.

Consequently, SSL3 protein can effectively be used as a vaccine against S. aureus induced mastitis.

### 5. Further Vaccination-Challenge Experiment with SSL3 Vaccination Against S. Aureus Induced Bovine Mastitis

A further vaccination-challenge experiment in cows was performed to investigate the timing of SSL3 vaccination. This experiment was essentially of the same design as that described in Examples 3 and 4, except that where Examples 3 and 4 applied vaccination during lactation (after calving), this experiment applied the vaccination at and around pregnancy. Heifers were vaccinated twice (at approximately 7 and 2 weeks) before calving (ergo: while pregnant), and once (at approximately 7 weeks) after calving. Intramammary challenge infection was at 4 weeks after the last vaccination (during lactation). Each group contained about 12 cows.

The vaccines tested were the same as used in Examples 3 and 4: an SSL3 comprising vaccine, and an empty mock vaccine.

Again the SSL3 comprising vaccine was found to provide protection against challenge: a reduction was observed of the number of S. aureus challenge bacteria that could be re-isolated out of the milk from the vaccinated group. Over a period of 6 weeks after challenge (10 time points) 28% of quarters was negative for re-isolation at any time point for the vaccinated animals, while only 14% for the mock vaccine group.

Also the somatic cell count (SCC) was reduced in the milk of vaccinated and challenge-infected quarters, when compared to mock-vaccinated challenge-infected quarters. In the period of 1 to 6 weeks after challenge, 15% of milk samples showed a SCC lower than 100,000 in the SSL3 vaccinated animals, whereas this was only 8% for the mock vaccinated group.

The cow sera from this experiment were also tested in competition-inhibition assays, to detect that SSL3-specific antibodies had been induced. The set-up and the performance of these were as described in Example 4.2 above, and the results are presented in FIG. 14, with panel A depicting the results of the mock-vaccinated sera (n=12), and panel B those of the sera from the SSL3 vaccinated cows (n=13).

Again, the last column of panel B (post-vac+SSL3 sample) indicated essentially no reduction of fluorescence intensity, indicating that specific anti-SSL3 antibodies had been formed in the SSL3 vaccinated cows, and these antibodies could prevent SSL3 protein from binding to TLR2.

The conclusion from comparing the favourable results from Examples 4 and 5 is that the vaccination with SSL3 in cows is apparently not dependent of the status of the cows: whether they are pregnant or not, and are lactating or not.

In all cases a vaccination with a vaccine containing SSL3 protein was capable of inducing specific anti-SSL3 antibodies, which antibodies prevented S. aureus SSL3 from interacting with a TLR2 receptor.

### LEGEND TO THE FIGURES

FIG. 1: Binding of SSL3-FITC to leukocytes

Leukocytes were incubated with 0, 1, 3 or 10 μg/ml FITC-labelled SSL3 for 30 min at 4° C. Neutrophils (A), monocytes (B), and lymphocytes (C) were gated according to forward- and side-scatter properties.

FIG. 2: SSL3 competes with antibody T2.5 for TLR2 binding

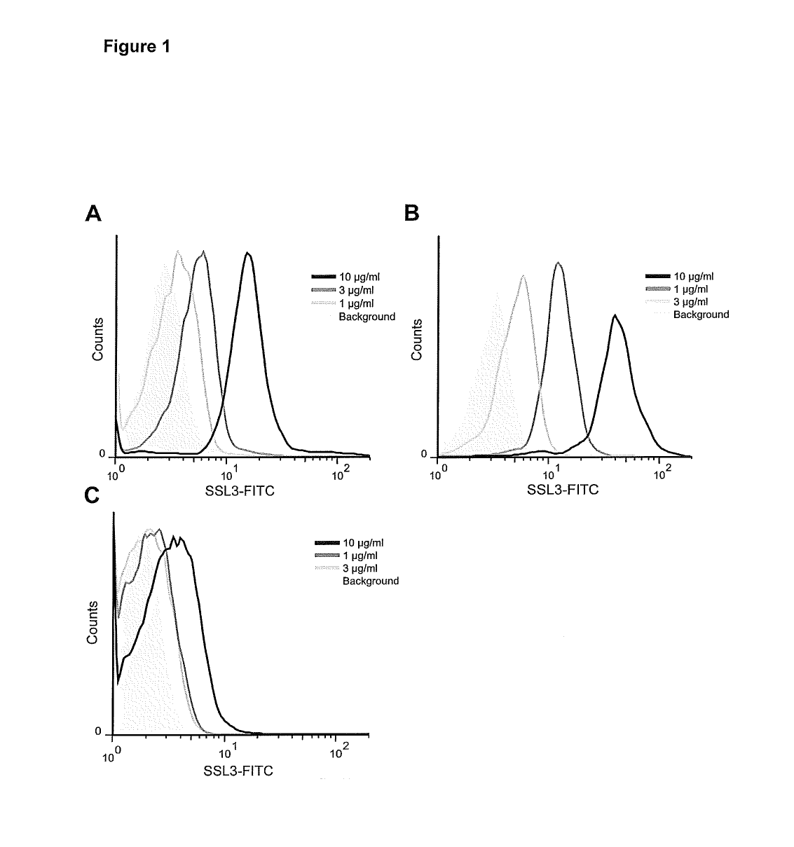

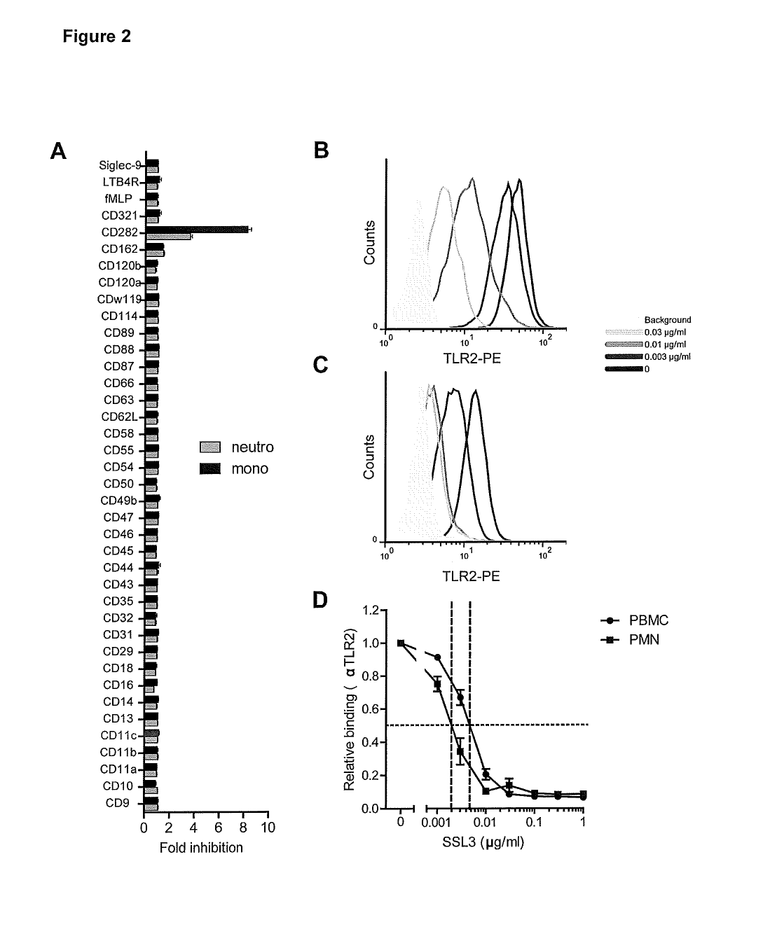

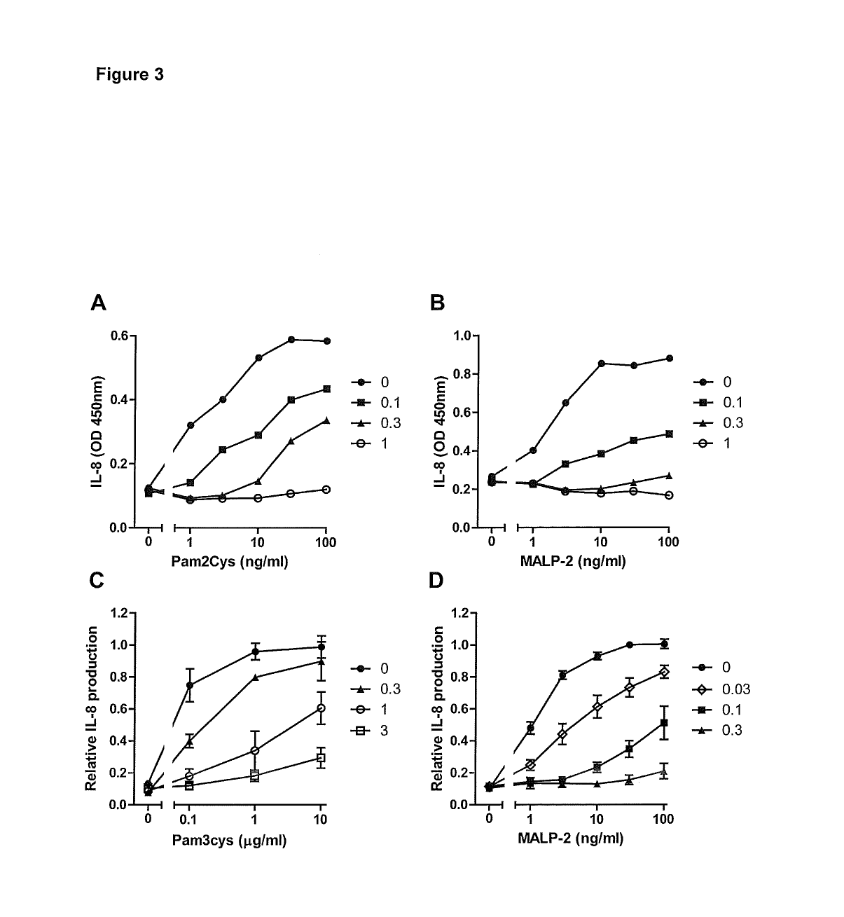

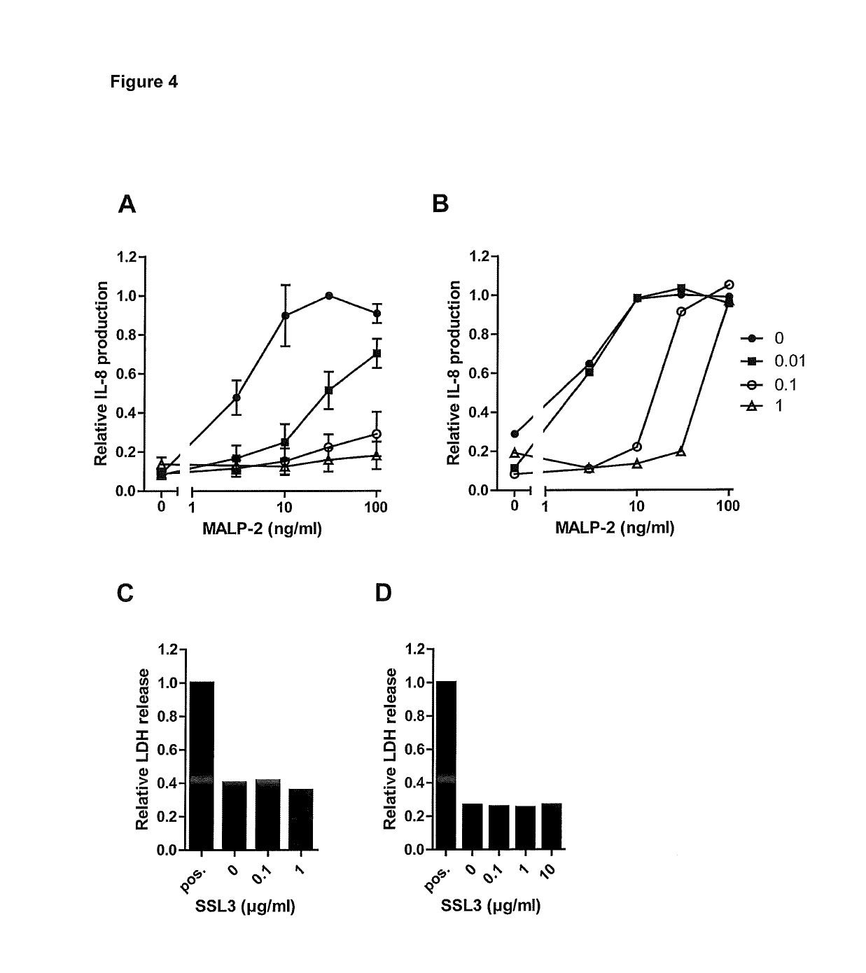

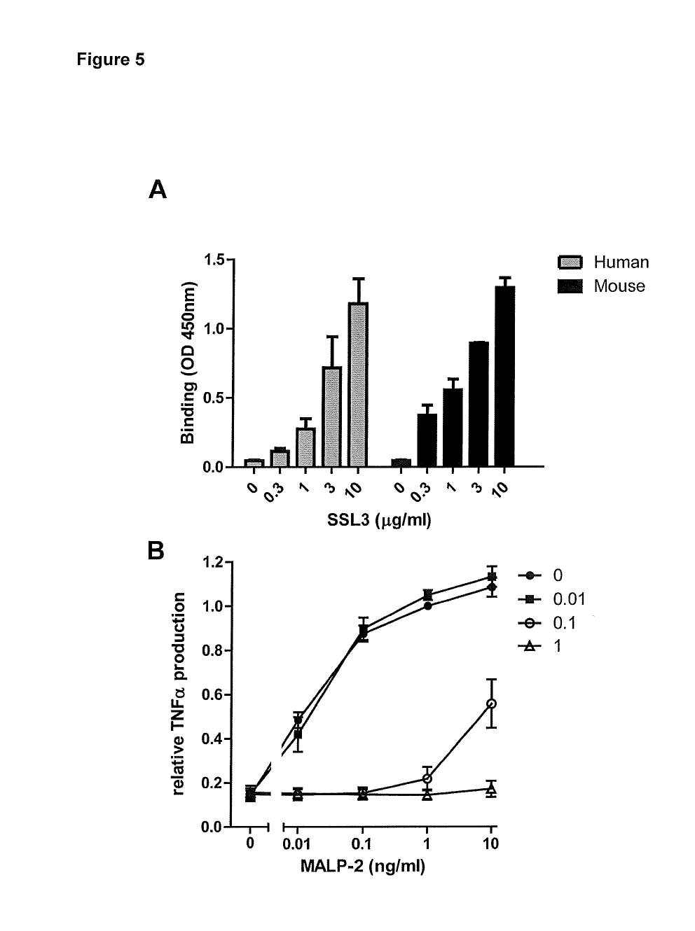

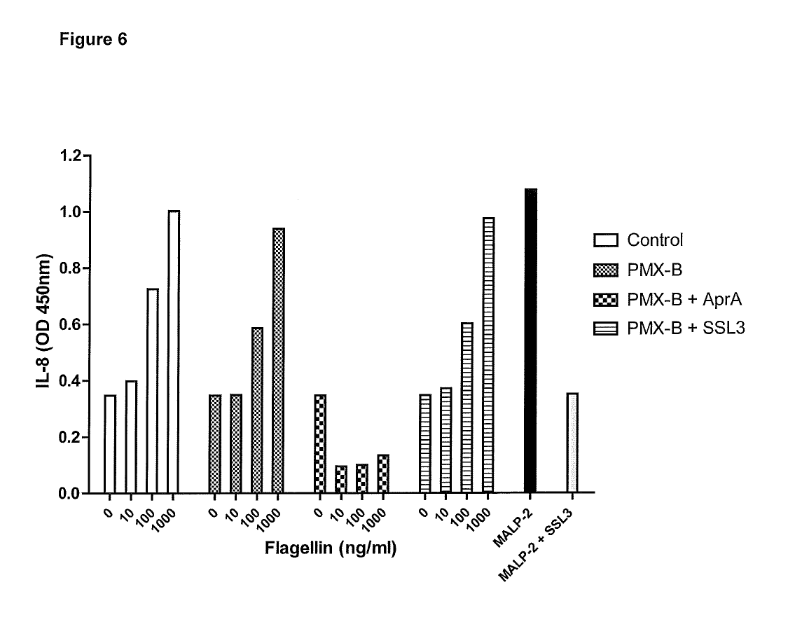

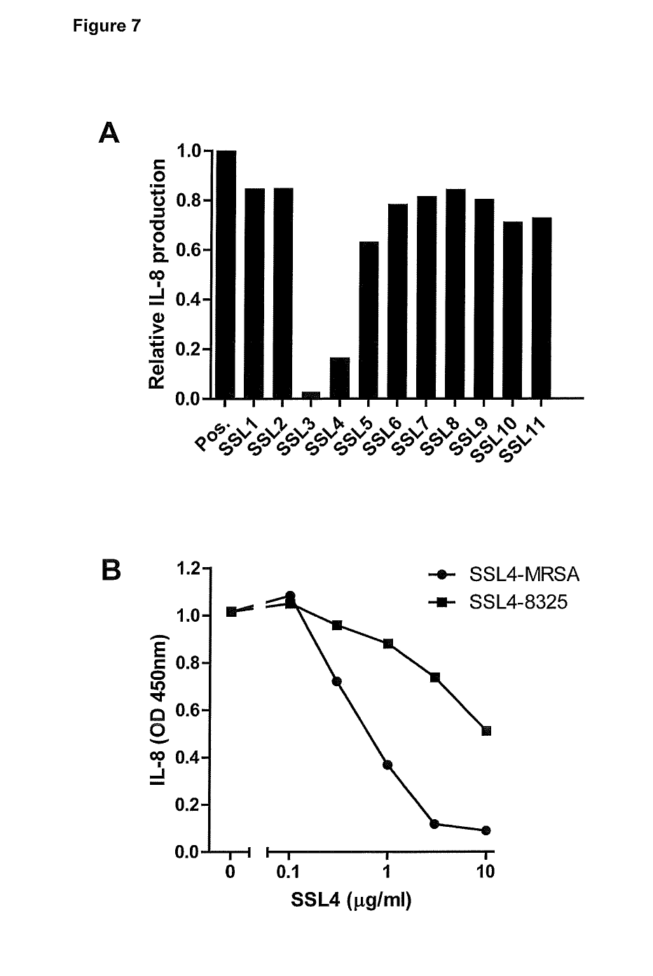

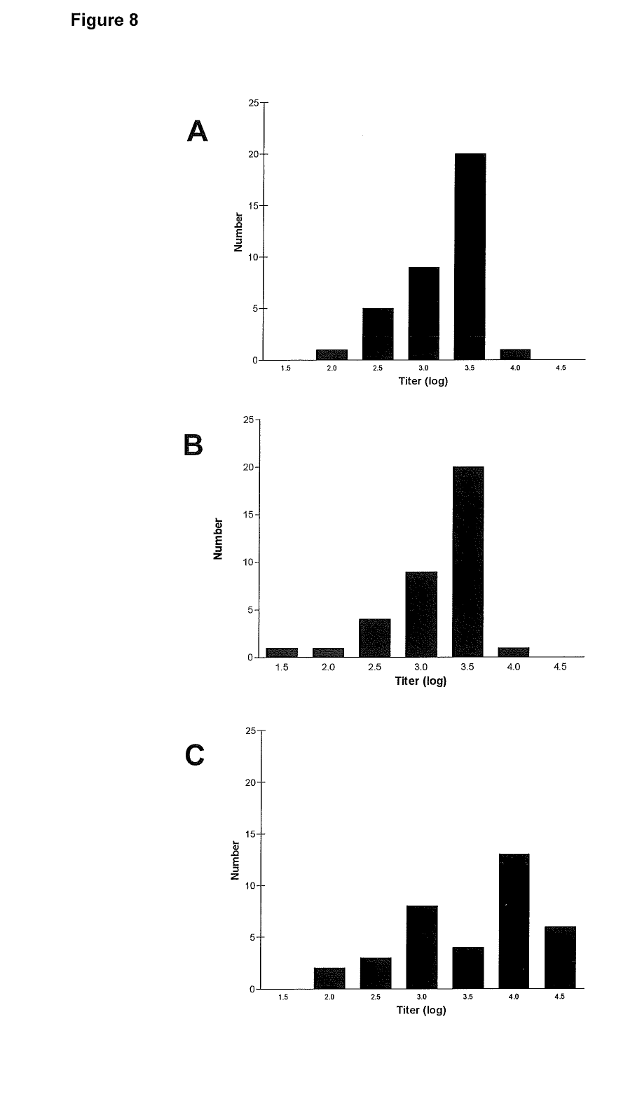

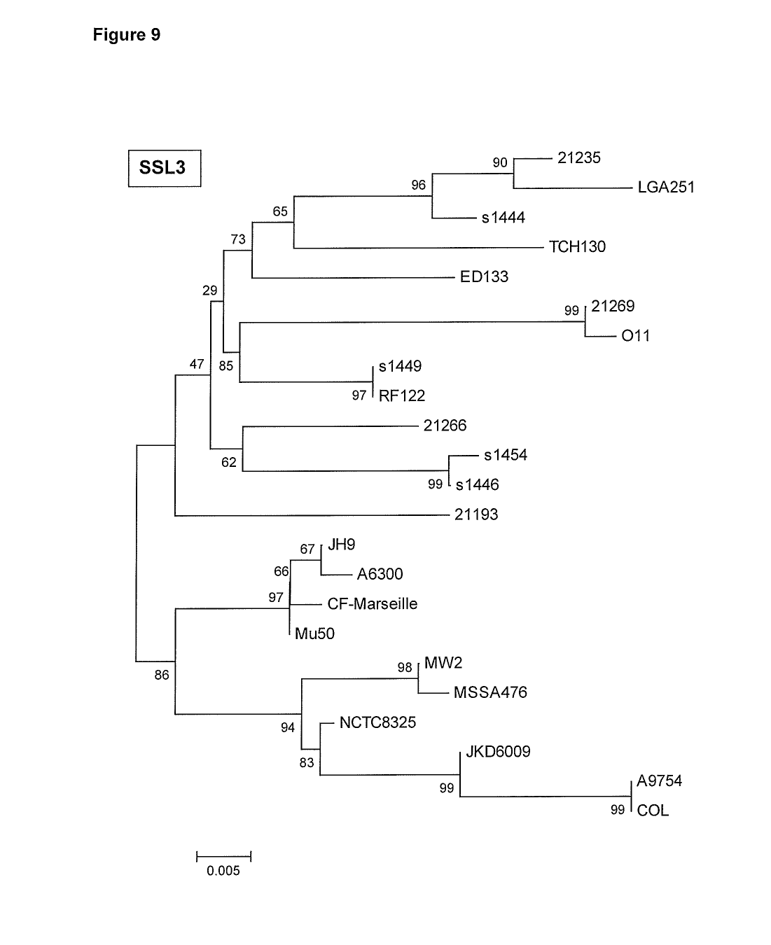

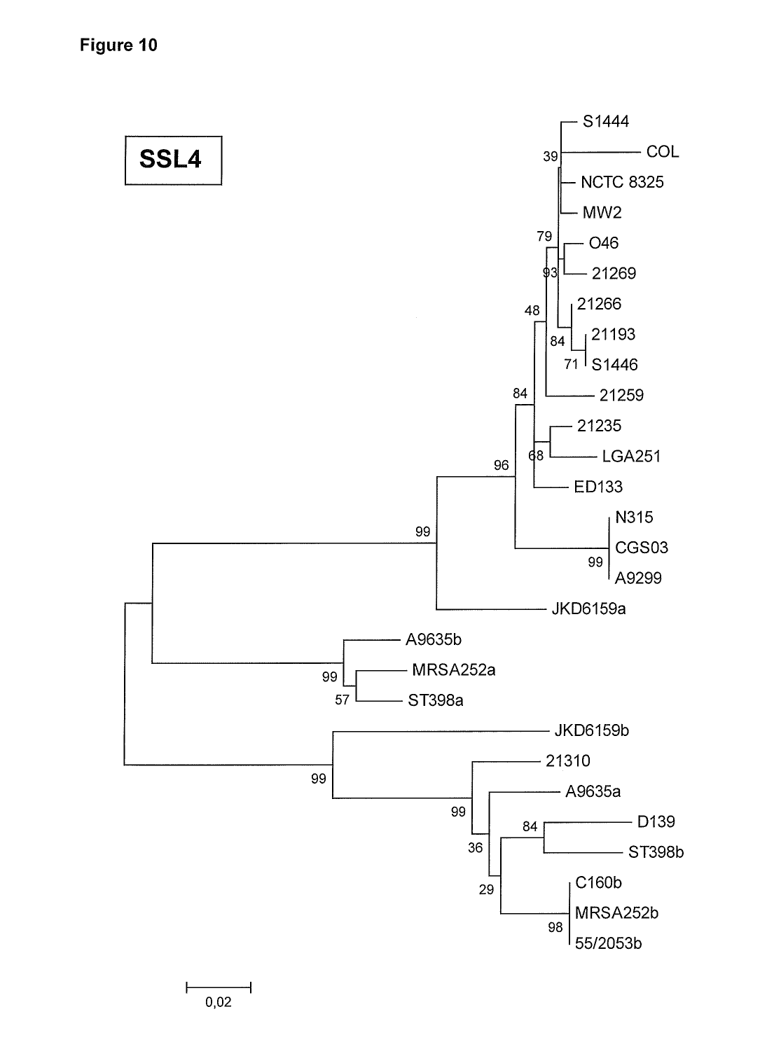

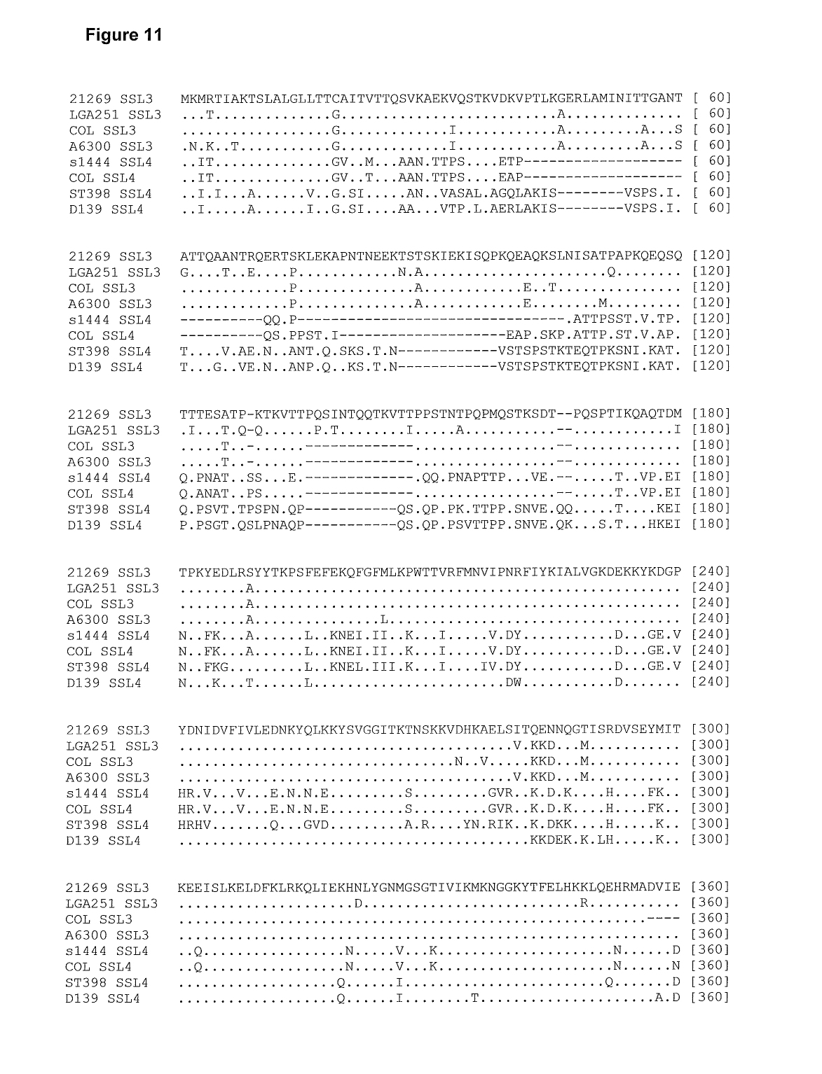

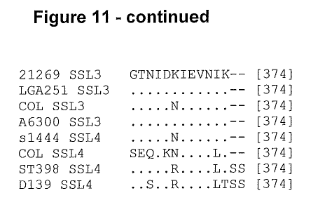

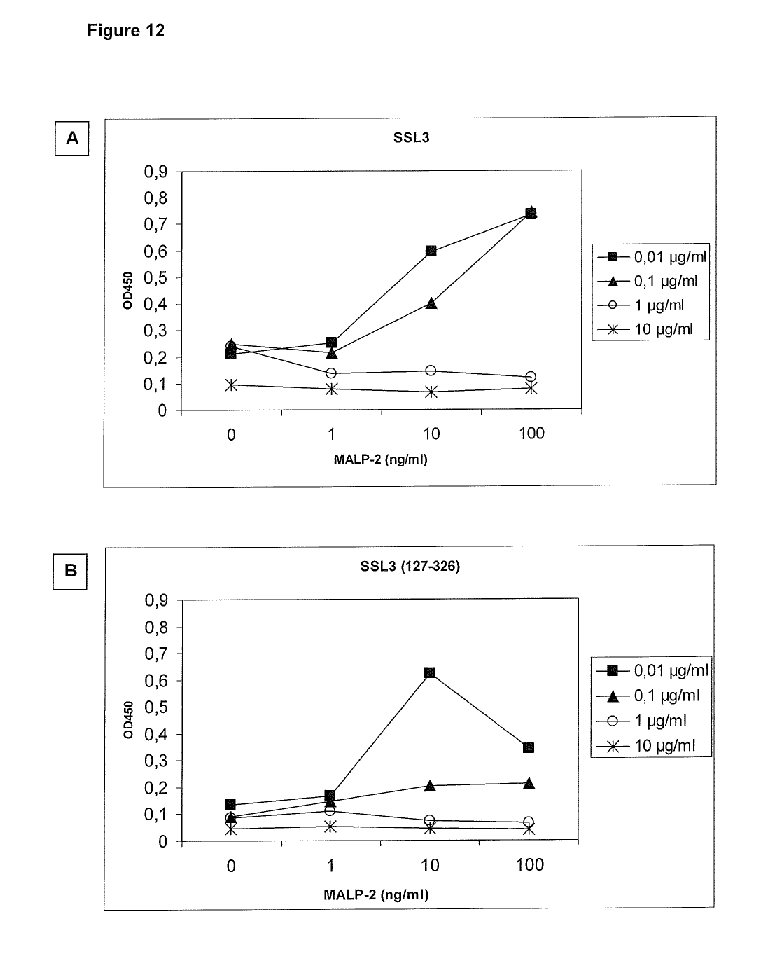

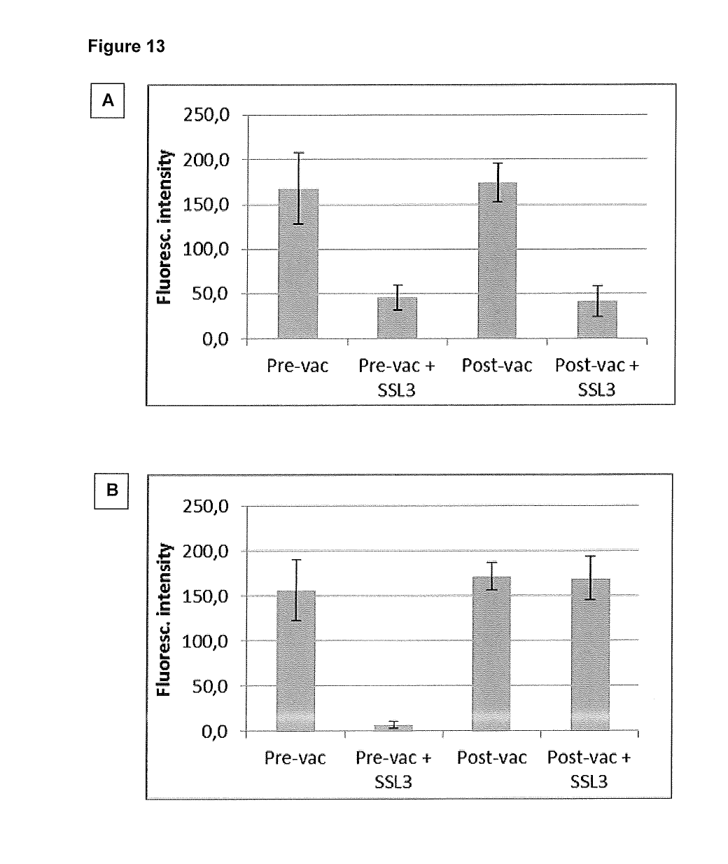

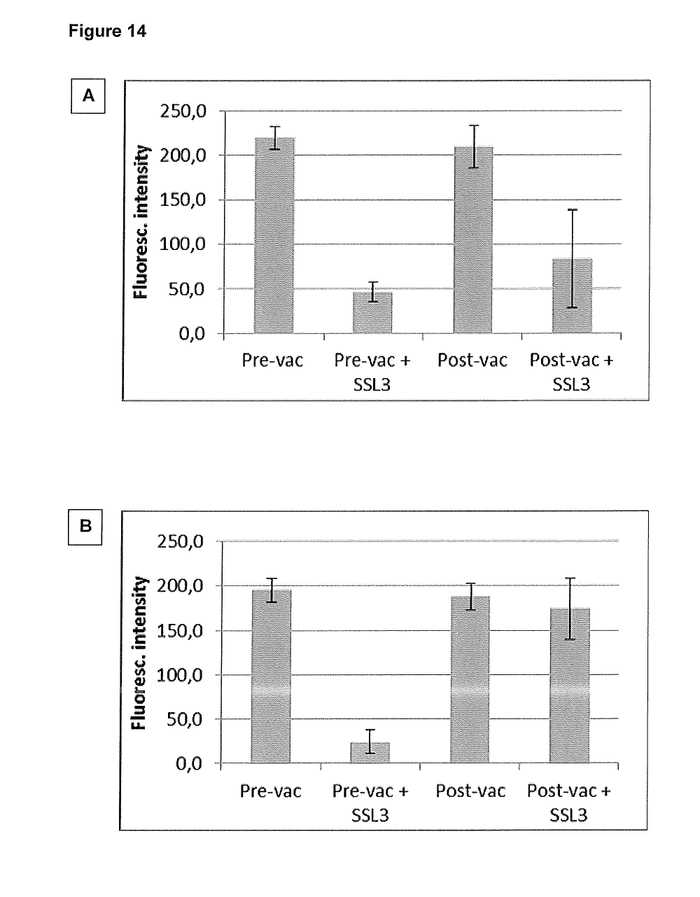

(A) Leukocytes were pre-incubated with 10 μg/ml SSL3 for 30 min at 4° C., and subsequently incubated with a panel of different monoclonal antibodies directed against cell-surface receptors for 30 min at 4° C. Fold inhibition was calculated by dividing the fluorescence of untreated cells by that of treated cells. Data represent mean±SEM of three independent experiments.

(B-D) Leukocytes were incubated with various concentrations of SSL3 for 30 min at 4° C. Next, cells were incubated with PE-labelled anti-TLR2 for 30 min at 4° C. Histograms depict binding of TLR2 to neutrophils (B) and monocytes (C). Relative fluorescence (D) of anti-TLR2 binding to neutrophils and monocytes to calculate the IC50. Data represent mean±SEM of three independent experiments.

FIG. 3: SSL3 inhibits the activation of TLR2 on HEK-TLR2 cells

(A, B) HEK cells transfected with TLR2 were incubated with 0, 0.1, 0.3 and 1 μg/ml SSL3 for 30 min. Cells were subsequently stimulated with increasing concentrations Pam2Cys (A) or MALP-2 (B).

(C) HEK-TLR1/2 were pre-incubated with 0, 0.1, 1, and 10 μg/ml SSL3 for 30 min, and subsequently stimulated with various concentrations Pam3Cys.

(D) HEK-TLR2/6 were pre-incubated with different concentrations SSL3 for 30 min, and subsequently stimulated with various concentrations MALP-2.

All stimulations were performed overnight and cell supernatant was collected to measure produced IL-8 levels by ELISA.

(A, B) IL-8 production is expressed as OD 450 nm.

(C) The IL-8 production relative to cells stimulated with 1 μg/ml Pam3Cys was calculated and expressed as mean±SD of triplicate experiments.

(D) The IL-8 production relative to cells stimulated with 30 ng/ml MALP-2 was calculated and expressed as mean±SEM of three independent experiments.

FIG. 4: SSL3 inhibits the activation of TLR2 on human leukocytes

(A, B) SSL3 was pre-incubated with 20 μg/ml polymyxin B and PBMCs were pre-incubated with 10 μg/ml anti-TLR4. Neutrophils (A) and PBMCs (B) were isolated from healthy donors and incubated with SSL3 for 30 min. Next, cells were stimulated with increasing concentrations of MALP-2. After overnight incubation, cell supernatant was harvested and IL-8 levels were determined by ELISA. Data are expressed as IL-8 production relative to stimulation with 30 ng/ml MALP-2. For neutrophils data represent mean±SEM of three independent experiments and for PBMCs a representative experiment is shown. (C, D) Analysis of cytotoxic effects of SSL3 on PBMCs (C) and HEK-TLR2/6 cells (D). Cells were incubated overnight with SSL3 and toxicity was tested using the lactate dehydrogenase (LDH) cellular cytotoxicity detection kit. LDH is depicted relative to the positive control (lysed cells).

FIG. 5: SSL3 binds to mouse TLR2 and functionally inhibits its activity

(A) A 96-wells plate was coated with the recombinant extracellular domain of mouse or human TLR2 (10 μg/ml). Coated wells were blocked with 4% skimmed milk, and subsequently increasing concentrations of His-SSL3 was added for 1 h at 37° C. Binding of SSL3 was detected with an anti-Xpress moab, followed by a peroxidase-labelled goat anti-mouse IgG. (B) Mouse macrophage cells (RAW264.7) were pre-incubated with SSL3 for 30 min. Next, cells were stimulated with increasing concentrations MALP-2. After overnight incubation, cell supernatant was collected and TNFα levels were determined by ELISA. Data are expressed as TNFα production relative to cells stimulated with 1 ng/ml MALP-2 and represent the mean±SEM of three independent experiments.

FIG. 6: TLR5 activation is not bound, and not inhibited by SSL3

Flagellin of P. aeruginosa was pre-incubated with polymyxin B (PMX-B; 20 μg/ml), PMX-B+AprA (10 μg/ml) or PMX-B+SSL3 (3 μg/ml) for 30 min at 37° C. Neutrophils were stimulated overnight with treated flagellin at 37° C. In addition, neutrophils were stimulated with MALP-2+/−SSL3 in the presence of PMX-B. Next, cell supernatant was collected and IL-8 production was measured by ELISA. Data are expressed as absorbance at 450 nm.

FIG. 7: Effect of other SSLs on inhibition of TLR2 activation

(A) HEK-TLR2/6 cells were pre-incubated with 10 μg/ml SSL1-11 for 30 min at 37° C., and subsequently stimulated with 3 ng/ml MALP-2. After overnight incubation, cell supernatant was harvested to determine IL-8 production by ELISA. IL-8 production is expressed relative to cells treated with MALP-2 only.

(B) HEK-TLR2/6 cells were pre-incubated with increasing concentrations of SSL4-8325 and SSL4-MRSA252 for 30 min, and subsequently stimulated with 30 ng/ml MALP-2. After overnight incubation, cell supernatant was collected and IL-8 production was determined by ELISA. Data are expressed as absorbance at 450 nm.

FIG. 8: Seroresponse against SSL3 and SSL4 in sera from healthy human volunteers

Results of an ELISA using sera from healthy human volunteers, on coated proteins: the SSL3 protein, the homolog, and the fragment, all for use according to the invention.

Data are presented as the frequency distribution of IgG titres measured. The titre was defined as the 10 log of the dilution that gave an absorbance of 0.400 relative Elisa units, after subtraction of background value.

FIG. 9: S. aureus SSL3 protein multiple alignment—graphic version

Most SSL3 amino acid sequences were retrieved from the public NCBI protein database, and some from non-public sequenced bovine S. aureus isolates. Partial SSL3 sequences were omitted from the further analysis, and for highly identical SSL3 proteins, only one representative sequence was used (see Table 2).

Sequences were aligned using the CLUSTALW™ program. The phylogenetic tree was constructed using the neighbour-joining method (with bootstrap 500) and evaluated using the interior branch test method with MEGA™ version 5 software (Tamura, Peterson, Stecher, Nei, and Kumar, 2011).

FIG. 10: S. aureus SSL4 protein multiple alignment—graphic version

See legend to FIG. 9, whereby FIG. 10 deals with SSL4 amino acid sequences (see Table 3).

FIG. 11: Multiple alignment of a representative number of S. aureus SSL3 and SSL4 proteins—text version.

Results from multiple amino acid sequence alignment using the ClustalW™ algorithm on the amino acid sequences from a representative selection of SSL3 and SSL4 proteins, each from 4 S. aureus isolates.

The protein sequences were derived from the NCBI database or from an in house sequencing program. The conserved amino acid residues are indicated by a dot; gaps in the sequence are indicated by a horizontal bar.

SSL3 is from strains: 21269, acc. no. EGS84524; LGA251, acc. no. CCC87131; COL, acc. no. YP_{—}185360; and A6300 acc. no. ZP_{—}05693238.

SSL4 is from strains: s1444, in house; COL, acc. no. YP_{—}185362; ST398, acc. no. CAQ48930; and D139, acc. no. ZP_{—}06323515.

FIG. 12: Inhibition of TLR2 by SSL3 and C-terminal fragment of SSL3

Similar to the results in FIG. 4, and performed according to Example 1.1.6, the inhibition of TLR2 activation, as detected by IL8 production, could be inhibited both by SSL3 (top-panel, A) and by a C-terminal fragment of SSL3, the amino acids 127-326 of SEQ ID NO:1 (bottom panel, B).

FIG. 13: Results from competition-inhibition assay

Sera from cows that were vaccinated with SSL3 protein (panel B) or mock-vaccinated (Panel A), according to the protocol of Examples 3 and 4, were tested from before- and after vaccination, and with- or without SSL3 protein, to detect presence of specific anti-SSL3 antibodies. Fluorescence intensities are given as average values with standard deviation.

FIG. 14: Results from further competition-inhibition assay

Similar to the presentation of FIG. 13, this figure presents the results from the competition-inhibition assay of the sera from the experiment outlined in Example 5, with sera from SSL3 protein vaccinated cows in panel B, and mock-vaccinated sera in panel A.

1-13. (canceled)

14. A vaccine against Staphylococcus aureus (S. aureus) comprising a Staphylococcal superantigen-like 3 (SSL3) protein, or a homolog of said SSL3 protein, or an immunogenic fragment of either protein, and an adjuvant.

15. The vaccine of claim 14, wherein the SSL3 protein is a protein comprising an amino acid sequence having at least 90% amino acid sequence identity to the amino acid sequence of SEQ ID NO. 1.

16. The vaccine of claim 14, wherein the homolog is a protein that is capable of direct binding to TLR2 and thereby inhibit the activation of the TIR domain of said TLR2 by a TLR2 ligand, and wherein said protein comprises an amino acid sequence having at least 56% amino acid sequence identity to the amino acid sequence of SEQ ID NO. 1.

17. The vaccine of claim 14, further comprising an antibody that can bind specifically to an SSL3 protein, or to a homolog of said SSL3 protein.

18. A vaccine against S. aureus comprising a nucleic acid encoding an SSL3 protein, or a homolog of said SSL3 protein, or an immunogenic fragment of either protein, and an adjuvant.

19. A vaccine against S. aureus comprising a live recombinant carrier micro-organism (LRCM), wherein said LRCM comprises a nucleic acid encoding an SSL3 protein, or a homolog of said SSL3 protein, or an immunogenic fragment of either protein, and an adjuvant.

20. The vaccine of claim 16, further comprising an antibody that can bind specifically to an SSL3 protein, or to a homolog of said SSL3 protein.

21. The vaccine of claim 15, further comprising an antibody that can bind specifically to an SSL3 protein, or to a homolog of said SSL3 protein.

22. A method for making the vaccine of claim 14, comprising the admixing of an SSL3 protein, or a homolog of said SSL3 protein, or an immunogenic fragment of either protein, and an adjuvant.

23. A method of vaccinating a human or animal subject, comprising the inoculating said human or animal subject with the vaccine of claim 14.

24. A method of vaccinating a human or animal subject, comprising the inoculating said human or animal subject with the vaccine of claim 18.

25. A method of vaccinating a human or animal subject, comprising the inoculating said human or animal subject with the vaccine of claim 17.

26. A method of vaccinating a human or animal subject, comprising the inoculating said human or animal subject with the vaccine of claim 16.

27. A method of vaccinating a human or animal subject, comprising the inoculating said human or animal subject with the vaccine of claim 15.


SEQUENCE LISTING (8 sequences)

SEQ ID NO: 1 - protein, 356 aa, Staphylococcus arlettae
  PEPTIDE (1)..(356): SSL3 - NCTC 8325 (YP_498973)
MKMRTIAKTSLALGLLTTGAITVTTQSVKAEKIQSTKVDKVPTLKAERLAMINITAGANSATTQAANTRQERTPKLEKAPNTNEEKTSASKIEKISQPKQEEQKTLNISATPAPKQEQSQTTTESTTPKTKVTTPPSTNTPQPMQSTKSDTPQSPTIKQAQTDMTPKYEDLRAYYTKPSFEFEKQFGFMLKPWTTVRFMNVIPNRFIYKIALVGKDEKKYKDGPYDNIDVFIVLEDNKYQLKKYSVGGITKTNSKKVNHKVELSITKKDNQGMISRDVSEYMITKEEISLKELDFKLRKQLIEKHNLYGNMGSGTIVIKMKNGGKYTFELHKKLQEHRMADVIDGTNIDNIEVNIK

SEQ ID NO: 2 - protein, 369 aa, Staphylococcus aureus
  PEPTIDE (1)..(369): SSL3 - Strain 1444
MKMRTIAKTSLALGLLTTGAITVTTQSVKAEKVQSTKVDKVPTLKAERLAMINITTGANTATTQAANTRQERTPKLEKAPNTNEKKNSASKIEKISQPKQEAQKSLNISATQAPKQEQSQTITESTTQQTKVTTPPSTNTQQTKVTIPPSTNAPQPMQSTKSDTPQSPTIKQAQTDITPKYEDLRAYYTKPSFEFEKQFGFMLKPWTTVRFMNVIPNRFIYKIALVGKDEKKYKDGPYDNIDVFIVLEDNKYQLKKYSVGGITKTNSKKVDHKAELSITKKDNQGMISRDVSEYMITKEEISLKELDFKLRKQLIEKHNLYGNMGSGTIVIKMKNGGKYTFELHKKLQEHRMADVIEGTNIDKIEVNIK

SEQ ID NO: 3 - protein, 356 aa, Staphylococcus aureus
  PEPTIDE (1)..(356): SSL3 - strain 1446
MKMRTIAKTSLALGLLTTGAITVTTQSVKAEKVQSTKVDKISTLKAERLAMINITAGANTVTTQAAKTGQERTPKLEKAPNTNEEKTSTSKIEKVSQPKQEAQKLLNISATPAPKQEQSQTTTESTTPKTRVTTPPSTNTPQPMQSTKSDTPQSPNIKQAQTDMTPKYEDLRAYYTKPSFEFEKQFGFMLKPWTTVRFMNVIPNRFIYKIALVGKDEKKYKDGPYDNIDVFIVLEDNKYQLKKYSVGGITKTNSKKVDHKAELSITKKDNQGMISRDVSEYMITKEEISLKELDFKLRKQLIEKHNLYGNMGSGTIVIKMKNGGKYTFELHKKLQEHRMADVIEGTNIDKIEVNIK

SEQ ID NO: 4 - protein, 348 aa, Staphylococcus aureus
  PEPTIDE (1)..(348): SSL3 - strain 1449
MKMRTIAKTSLALGLLTTGAITVTTQSVKAEKVPMLKAERLAMINITTGANTATTQAANTRQERTPKLEKAPNTNEEKTSASKIEKISQPKQEAQKSLNISATPAPKQEQSQTTTESTTPKTKVTTPPSINTPQPMQSTKSDTPQSPTIKQAQTDMTPKYEDLRAYYTKPSFEFEKQFGFMLKPWTTVRFMNVIPNRFIYKIALVGKDEKKYKDGPYDNIDVFIVLEDNKYQLKKYSVGGITKTNSKKVDHKAELSITKKDNQGMISRDVSEYMITKEEISLKELDFKLRKQLIEKHNLYGNMGSGTIVTKMKNGGKYTFELHKKLQEHRMADVIEGTNIDKIEVNIK

SEQ ID NO: 5 - protein, 356 aa, Staphylococcus aureus
  PEPTIDE (1)..(356): SSL3 - strain 1454
MKMRTIAKTSLALGLLTTGAITVTTQSVKAEKVQSTKVDKISTLKAERLAMINITAGANTVTTQAAKTGQERTPKLEKAPNTNEEKTSTSKIEKVSQPKQEAQKLLNISATPAPKQEQSQTTTESTTPKTRVTTPPSTNTPQPMQSTKSDTPQSPNIKQAQTDMTPKYEDLRAYYTKPSFEFEKQFGFMLKPWTTVRFMNVIPNRFIYKIALVGKDEKKYKDGPYDNIDVFIVLEDNKYQLKKYSVGGITKTNSKKVDHKAELSITKKDNQGMISRDVSEYMITKEEISLKELDFKLRKQLIEKHNLYGNMGSGTIVIKMKNGGKYTFELHKKLQEHRMADVIEVTNIDKIEVNIK

SEQ ID NO: 6 - protein, 308 aa, Staphylococcus aureus
  PEPTIDE (1)..(308): SSL4 - NCTC 8325 (YP_498975)
MKITTIAKTSLALGLLTTGVITTTTQAANATTLSSTKVEAPQSTPPSTKIEAPQSKPNATTPPSTKVEAPQQTANATTPPSTKVTTPPSTNTPQPMQSTKSDTPQSPTTKQVPTEINPKFKDLRAYYTKPSLEFKNEIGIILKKWTTIRFMNVVPDYFIYKIALVGKDDKKYGEGVHRNVDVFVVLEENNYNLEKYSVGGITKSNSKKVDHKAGVRITKEDNKGTISHDVSEFKITKEQISLKELDFKLRKQLIEKNNLYGNVGSGKIVIKMKNGGKYTFELHKKLQENRMADVIDGTNIDNIEVNIK

SEQ ID NO: 7 - protein, 296 aa, Staphylococcus aureus
  PEPTIDE (1)..(296): SSL4 - strain 1444
MKITTIAKTSLALGLLTTGVITMTTQAANATTPSSTKVETPQQTPNATTPSSTKVETPQQTPNATTPSSTKVETPQQTPNAPTTPSTKVETPQSPTTKQVPTEINPKFKDLRAYYTKPSLEFKNEIGIILKKWTTIRFMNVVPDYFIYKIALVGKDDKKYGEGVHRNVDVFVVLEENNYNLEKYSVGGITKSNSKKVDHKAGVRITKEDNKGTISHDVSEFKITKEQISLKELDFKLRKQLIEKNNLYGNVGSGKIVIKMKNGGKYTFELHKKLQENRMADVIDGTNIDNIEVNIK

SEQ ID NO: 8 - protein, 293 aa, Staphylococcus aureus
  PEPTIDE (1)..(293): SSL4 - strain 1446
MKITTIAKTSLALGLLTTGVITTTTQAANATTPSSTKVEAPQQTANATTPSSTKVEVPQSTPLSTKVEAPQSKPNATTPPSSNVDTSPPQSPTTKQVPTEINPKFKDLRAYYTKPSIEFKNEIGIILKKWTTIRFMNVVPDYFIYKIALVGKDDKKYGEGVHRNVDVFVVLEENNYNLEKYSVGGITKSNSKKVDHKAGVRITKEDNKGIISHDVSEFKITKEQISLKELDFKLRKQLIEKNNLYGNVGSGKIVIKMKNGGKYTFELHKKLQENRMADVIDGTNIDNIEVNIK